(19) United States
(12) Patent Application Publication
(10) Pub. No.: US 2023/0365572 A1
(43) Pub. Date: Nov. 16, 2023

(54) HYDROXYLBENZYL AZABICYCLO[2.2.1]HEPTAN-1-ANE DRIVATIVES AND MEDICAL USES THEREOF

(71) Applicant: ATROGI AB, Stockholm (SE)

(72) Inventors: Ilga Mutule, Jurmala (LV); Benjamin Pelcman, Stockholm (SE); Tore Bengtsson, Vaxholm (SE)

(21) Appl. No.: 18/027,890

(22) PCT Filed: Sep. 23, 2021
(86) PCT No.: PCT/EP2021/076185

§ 371 Date: Mar. 22, 2023

(30) Foreign Application Priority Data
Sep. 23, 2020 (GB) 2015035.5

(51) Int. Cl. C07D 487/08

(52) U.S. Cl. CPC C07D 487/08

(57) ABSTRACT

There is provided herein a compound of formula I or a pharmaceutically acceptable salt thereof, wherein R¹ and the ring comprising Q¹ to Q⁵ have meanings provided in the description.

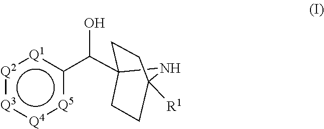

### FIELD OF THE INVENTION

The present invention relates to novel compounds and compositions, and their use in the treatment of hyperglycaemia and disorders characterised by hyperglycaemia, such as type 2 diabetes. In particular, the invention relates to novel compounds, compositions and methods for the treatment of conditions such as type 2 diabetes through activation of the B2-adrenergic receptor. Importantly, such compounds are thought to have a beneficial side-effect profile as they do not exert their effect through significant cAMP release.

### BACKGROUND OF THE INVENTION

The listing or discussion of an apparently prior-published document in this specification should not necessarily be taken as an acknowledgement that the document is part of the state of the art or is common general knowledge.

Hyperglycaemia, or high blood sugar is a condition in which an excessive amount of glucose circulates in the blood plasma. If not treated, hyperglycaemia can be a serious problem, potentially developing into life-threatening conditions such as ketoacidosis. For example, chronic hyperglycemia may cause injury to the heart, and is strongly associated with heart attacks and death in subjects with no coronary heart disease or history of heart failure. There are various causes of hyperglycaemia, including diabetes and severe insulin resistance.

Severe insulin resistance (SIR) is a condition wherein the patent experiences very low levels of (or, in extreme cases, no significant) response to insulin. There are several syndromes characterized by SIR, including Rabson-Mendenhall syndrome, Donohue's syndrome (leprechaunism), Type A and Type B syndromes of insulin resistance, the HAIR-AN (hyperandrogenism, insulin resistance, and acanthosis nigricans) syndrome, pseudoacromegaly, and lipodystrophy. The majority of these conditions have genetic causes, such as mutations in the insulin receptor gene. The prevalence for Donohue's syndrome, Rabson-Mendenhall syndrome and Type A syndrome of insulin resistance, has been reported to vary from about 50 reported cases to 1 in 100,000. However, since some diseases are severe and extremely rare, it is likely that many patients do not get diagnosed before they die, particularly in less developed areas of the world. Thus, the exact number of patients with these syndromes is difficult to assess.

The current standard for hyperglycaemia treatment in patients having SIR is a controlled diet, supplemented with drugs affecting insulin receptor sensitivity, such as metformin, or insulin supplement. However, particularly for disorders caused by mutations in the insulin receptor gene, this treatment is not sufficiently effective and ultimately proves unsuccessful.

Diabetes comprises two distinct diseases, type 1 (or insulin-dependent diabetes) and type 2 (insulin-independent diabetes), both of which involve the malfunction of glucose homeostasis. Type 2 diabetes affects more than 400 million people in the world and the number is rising rapidly. Complications of type 2 diabetes include severe cardiovascular problems, kidney failure, peripheral neuropathy, blindness and, in the later stages of the disease, even loss of limbs and, ultimately death. Type 2 diabetes is characterized by insulin resistance in skeletal muscle and adipose tissue, and there is presently no definitive cure. Most treatments used today are focused on remedying dysfunctional insulin signalling or inhibiting glucose output from the liver but many of those treatments have several drawbacks and side effects. There is thus a great interest in identifying novel insulin-independent ways to treat type 2 diabetes.

In type 2 diabetes, the insulin-signalling pathway is blunted in peripheral tissues such as adipose tissue and skeletal muscle. Methods for treating type 2 diabetes typically include lifestyle changes, as well as insulin injections or oral medications to regulate glucose homeostasis. People with type 2 diabetes in the later stages of the disease develop ‘beta-cell failure’ i.e. the inability of the pancreas to release insulin in response to high blood glucose levels. In the later stages of the disease patients often require insulin injections in combination with oral medications to manage their diabetes. Further, most common drugs have side effects including downregulation or desensitization of the insulin pathway and/or the promotion of lipid incorporation in adipose tissue, liver and skeletal muscle. There is thus a great interest in identifying novel ways to treat metabolic diseases including type 2 diabetes that do not include these side effects.

Following a meal, increased blood glucose levels stimulate insulin release from the pancreas. Insulin mediates normalization of the blood glucose levels. Important effects of insulin on glucose metabolism include facilitation of glucose uptake into skeletal muscle and adipocytes, and an increase of glycogen storage in the liver. Skeletal muscle and adipocytes are responsible for insulin-mediated glucose uptake and utilization in the fed state, making them very important sites for glucose metabolism.

The signalling pathway downstream from the insulin receptor has been difficult to understand in detail. In brief, control of glucose uptake by insulin involves activation of the insulin receptor (IR), the insulin receptor substrate (IRS), the phosphoinositide 3-kinase (PI3K) and thus stimulation of phosphatidylinositol (3,4,5)-triphosphate (PIP3), the mammalian target of rapamycin (also called the mechanistic target of rapamycin, mTOR), Akt/PKB (Akt) and TBC1D4 (AS160), leading to translocation of the glucose transporter 4 (GLUT4) to the plasma membrane. Akt activation is considered necessary for GLUT4 translocation.

It should be noted that skeletal muscles constitute a major part of the body weight of mammals and have a vital role in the regulation of systemic glucose metabolism, being responsible for up to 85% of whole-body glucose disposal. Glucose uptake in skeletal muscles is regulated by several intra- and extracellular signals. Insulin is the most well studied mediator but others also exist. For example, AMP activated kinase (AMPK) functions as an energy sensor in the cell, which can increase glucose uptake and fatty acid oxidation. Due to the great influence skeletal muscles have on glucose homeostasis it is plausible that additional mechanisms exist. In the light of the increased prevalence of type 2 diabetes, it is of great interest to find and characterize novel insulin-independent mechanisms to increase glucose uptake in muscle cells.

Blood glucose levels may be regulated by both insulin and catecholamines, but they are released in the body in response to different stimuli. Whereas insulin is released in response to the rise in blood sugar levels (e.g. after a meal), epinephrine and norepinephrine are released in response to various internal and external stimuli, such as exercise, emotions and stress, and also for maintaining tissue homeostasis. Insulin is an anabolic hormone that stimulates many processes involved in growth including glucose uptake, glycogen and triglyceride formation, whereas catecholamines are mainly catabolic.

Although insulin and catecholamines normally have opposing effects, it has been shown that they have similar actions on glucose uptake in skeletal muscle (Nevzorova et al., Br. J. Pharmacol, 137, 9, (2002)). In particular, it has been reported that catecholamines stimulate glucose uptake via adrenergic receptors (Nevzorova et al., Br. J. Pharmacol, 147, 446, (2006); Hutchinson, Bengtsson Endocrinology 146, 901, (2005)) to supply muscle cells with an energy-rich substrate. Thus it is likely that in mammals, including humans, the adrenergic and the insulin systems can work independently to regulate the energy needs of skeletal muscle in different situations. Since insulin also stimulates many anabolic processes, including some that promote undesired effects such as stimulation of lipid incorporation into tissues, leading to e.g. obesity, it would be beneficial to be able to stimulate glucose uptake by other means; for example, by stimulation of the adrenergic receptors (ARs).

All ARs are G protein-coupled receptors (GPCRs) located in the cell membrane and characterized by an extracellular N-terminus, followed by seven transmembrane α-helices (TM-1 to TM-7) connected by three intracellular (IL-1 to IL-3) and three extracellular loops (EL-1 to EL-3), and finally an intracellular C-terminus. There are three different classes of ARs, with distinct expression patterns and pharmacological profiles: α₁-, α₂- and β-ARs. The α₁-ARs comprise the α_{1A}, α_{1B} and α_{1D} subtypes while α₂-ARs are divided into α_{2A}, α_{2B} and α_{2C}. The β-ARs are also divided into the subtypes β₁, β₂, and β₃, of which β₂-AR is the major isoform in skeletal muscle cells. ARs are G protein coupled receptors (GPCRs) that signal through classical secondary messengers such as cyclic adenosine monophosphate (cAMP) and phospholipase C (PLC).

Many effects occurring downstream of ARs in skeletal muscles have been attributed to classical secondary messenger signalling, such as increase in cAMP levels, PLC activity and calcium levels. Stimulation involving the classical secondary messengers has many effects in different tissues. For example, it increases heart rate, blood flow, airflow in lungs and release of glucose from the liver, which all can be detrimental or be considered unwanted side effects if stimulation of ARs should be considered as a type 2 diabetes treatment. Adverse effects of classical AR agonists are, for example, tachycardia, palpitation, tremor, sweats, agitation and increased glucose levels in the blood (glucose output from the liver). It would thus be beneficial to be able to activate ARs without activating these classical secondary messengers, such as cAMP, to increase glucose uptake in peripheral tissues without stimulating the unwanted side effects.

Glucose uptake is mainly stimulated via facilitative glucose transporters (GLUT) that mediate glucose uptake into most cells. GLUTs are transporter proteins that mediate transport of glucose and/or fructose over the plasma membrane down the concentration gradient. There are fourteen known members of the GLUT family, named GLUT1-14, divided into three classes (Class I, Class II and Class III) dependent on their substrate specificity and tissue expression. GLUT1 and GLUT4 are the most intensively studied isoforms and, together with GLUT2 and GLUT3, belong to Class I which mainly transports glucose (in contrast to Class II that also transports fructose). GLUT1 is ubiquitously expressed and is responsible for basal glucose transport. GLUT4 is only expressed in peripheral tissues such as skeletal muscle, cardiac muscle and adipose tissues. GLUT4 has also been reported to be expressed in, for example, the brain, kidney, and liver. GLUT4 is the major isoform involved in insulin stimulated glucose uptake. The mechanism whereby insulin signalling increases glucose uptake is mainly via GLUT4 translocation from intracellular storage to the plasma membrane. It is known that GLUT4 translocation is induced by stimulation of the β₂-adrenergic receptor.

Thus, a possible treatment of a condition involving dysregulation of glucose homeostasis or glucose uptake in a mammal, such as type 2 diabetes, would involve the activation of the β₂-adrenergic receptor leading to GLUT4 translocation to the plasma membrane and promotion of glucose uptake into skeletal muscle leading to normalization of whole body glucose homeostasis. In addition, it would be advantageous if the treatment does not involve signalling through cAMP as this would lead to a favourable side-effect profile.

### DESCRIPTION OF THE INVENTION

We have now surprisingly found that certain hydroxylbenzyl azabicyclo[2.2.1]heptan-1-anes acting as agonists at the β₂-adrenergic receptor increase glucose uptake in skeletal muscle.

In addition, we have found that this effect is not mediated through significant cAMP release, such that many of the commonly described side effects seen with traditional β₂-adrenergic agonists (e.g. tachycardia, palpitation, tremor, sweats, agitation, and the like) can be reduced.

The use of such compounds in medicine represents a promising strategy for the treatment of conditions characterized by high blood sugar levels (i.e. hyperglycaemia), such as type 2 diabetes.

Compounds of the Invention

In a first aspect of the invention, there is provided a compound of formula I

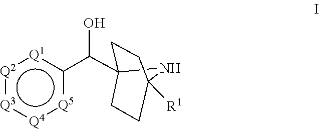

or a pharmaceutically acceptable salt thereof, wherein:
the ring comprising Q¹ to Q⁵ represents:
a phenyl optionally substituted with one or more X¹; or
a 5- or 6-membered heteroaryl optionally substituted with one or more X²;

each X¹ and X² independently represents halo, R^{a}, —CN, —N₃, —N(R^{b})R^{c}, —NO₂, —ONO₂, —OR^{d}, —S(O)ₚR^{e} or —S(O)_{q}N(R^{f}R^{g});

each R^{a} and R^{e} represents C₁₋₆ alkyl, C₂₋₆ alkenyl or C₂₋₆ alkynyl optionally substituted by one or more groups independently selected from G¹;

each R^{b}, R^{c}, R^{d}, R^{f} and R^{g} independently represents H or C₁₋₆ alkyl, C₂₋₆ alkenyl or C₂₋₆ alkynyl optionally substituted by one or more groups independently selected from G²;

or alternatively any of R^{b} and R^{c} and/or R^{f} and R^{g} may be linked together to form, together with the nitrogen atom to which they are attached, a 4- to 6-membered ring, which ring optionally contains one further heteroatom and which ring optionally is substituted by one or more groups independently selected from halo, C₁₋₃ alkyl optionally substituted by one or more halo, and ═O;

R¹ represents linear or branched C₁₋₁₂ alkyl, linear or branched C₂₋₁₂ alkenyl or linear or branched C₂₋₁₂ alkynyl, each optionally substituted by one G³;

each G¹ and G² independently represents halo, —CN, —N(R^{a1})R^{b1}, —OR^{c1}, —S(O)ₚR^{d1}, —S(O)_{q}N(R^{e1})R^{f1} or ═O;

G³ represents —OR^{h1};

each R^{a1}, R^{b1}, R^{c1}, R^{e1} and R^{f1} independently represents H or C₁₋₆ alkyl, C₂₋₆ alkenyl or C₂₋₆ alkynyl optionally substituted by one or more halo;

R^{d1} represents C₁₋₆ alkyl, C₂₋₆ alkenyl or C₂₋₆ alkynyl optionally substituted by one or more halo;

or alternatively any of R^{a1} and R^{b1} and/or R^{e1} and R^{f1} may be linked together to form, together with the nitrogen atom to which they are attached, a 4- to 6-membered ring, which ring optionally contains one further heteroatom and which ring optionally is substituted by one or more groups independently selected from halo, C₁₋₃ alkyl optionally substituted by one or more halo, and ═O;

R^{h1} represents

C₁₋₆ alkyl, or

aryl or heteroaryl optionally substituted by one or more halo;

each p independently represents 0, 1 or 2;

each q independently represents 1 or 2,

which compounds (including pharmaceutically acceptable salts) may be referred to herein as “compounds of the invention”.

For the avoidance of doubt, the skilled person will understand that references herein to compounds of particular aspects of the invention (such as the first aspect of the invention, e.g. compounds of formula I) will include references to all embodiments and particular features thereof, which embodiments and particular features may be taken in combination to form further embodiments.

Unless indicated otherwise, all technical and scientific terms used herein have the same meaning as commonly understood by one of ordinary skill in the art to which this invention pertains.

Pharmaceutically acceptable salts include acid addition salts and base addition salts. Such salts may be formed by conventional means, for example by reaction of a free acid or a free base form of a compound of the invention with one or more equivalents of an appropriate acid or base, optionally in a solvent, or in a medium in which the salt is insoluble, followed by removal of said solvent, or said medium, using standard techniques (e.g. in vacuo, by freeze-drying or by filtration). Salts may also be prepared by exchanging a counter-ion of a compound of the invention in the form of a salt with another counter-ion, for example using a suitable ion exchange resin.

Particular acid addition salts that may be mentioned include carboxylate salts (e.g. formate, acetate, trifluoroacetate, propionate, isobutyrate, heptanoate, decanoate, caprate, caprylate, stearate, acrylate, caproate, propiolate, ascorbate, citrate, glucuronate, glutamate, glycolate, a-hydroxybutyrate, lactate, tartrate, phenylacetate, mandelate, phenylpropionate, phenylbutyrate, benzoate, chlorobenzoate, methyl benzoate, hydroxybenzoate, methoxybenzoate, dinitrobenzoate, o-acetoxy-benzoate, salicylate, nicotinate, isonicotinate, cinnamate, oxalate, malonate, succinate, suberate, sebacate, fumarate, malate, maleate, hydroxymaleate, hippurate, phthalate or terephthalate salts), halide salts (e.g. chloride, bromide or iodide salts), sulphonate salts (e.g. benzenesulphonate, methyl-, bromo- or chloro-benzenesulphonate, xylenesulphonate, methanesulphonate, ethanesulphonate, propanesulphonate, edisylate, hydroxy-ethanesulphonate, 1- or 2-naphthalene-sulphonate or 1,5-naphthalenedisulphonate salts) or sulphate, pyrosulphate, bisulphate, sulphite, bisulphite, phosphate, monohydrogenphosphate, dihydrogenphosphate, metaphosphate, pyrophosphate or nitrate salts, and the like.

Particular acid addition salts that may be mentioned include the fumerate, maleate, succinate and hydrochloride (HCl) salt.

For the avoidance of doubt, the skilled person will understand that acid addition salts may include diacid salts (e.g. dihydrochloride salts).

Particular base addition salts that may be mentioned include salts formed with alkali metals (such as Na and K salts), alkaline earth metals (such as Mg and Ca salts), organic bases (such as ethanolamine, diethanolamine, triethanolamine, tromethamine and lysine) and inorganic bases (such as ammonia and aluminium hydroxide). More particularly, base addition salts that may be mentioned include Mg, Ca and, most particularly, K and Na salts.

For the avoidance of doubt, compounds of the first aspect of the invention may exist as solids, and thus the scope of the invention includes all amorphous, crystalline and part crystalline forms thereof, and may also exist as oils. Where compounds of the first aspect of the invention exist in crystalline and part crystalline forms, such forms may include solvates, which are included in the scope of the invention. Compounds of the first aspect of the invention may also exist in solution.

Compounds of the first aspect of the invention may contain double bonds and may thus exist as E (entgegen) and Z (zusammen) geometric isomers about each individual double bond. All such isomers and mixtures thereof are included within the scope of the invention.

Compounds of the first aspect of the invention may also exhibit tautomerism. All tautomeric forms and mixtures thereof are included within the scope of the invention.

Compounds of the first aspect of the invention may also contain more than one asymmetric carbon atoms and may therefore exhibit optical and/or diastereoisomerism. Diastereoisomers may be separated using conventional techniques, e.g. chromatography or fractional crystallisation. The various stereoisomers (i.e. enantiomers) may be isolated by separation of a racemic or other mixture of the compounds using conventional, e.g. fractional crystallisation or HPLC, techniques. Alternatively the desired optical isomers may be obtained from appropriate optically active starting materials under conditions which will not cause racemisation or epimerisation (i.e. a ‘chiral pool’ method), by reaction of the appropriate starting material with a ‘chiral auxiliary’ which can subsequently be removed at a suitable stage, by derivatisation (i.e. a resolution, including a dynamic resolution); for example, with a homochiral acid followed by separation of the diastereomeric derivatives by conventional means such as chromatography, or by reaction with an appropriate chiral reagent or chiral catalyst all under conditions known to the skilled person. All stereoisomers and mixtures thereof are included within the scope of the invention.

As used herein, references to halo and/or halogen groups will each independently refer to fluoro, chloro, bromo and iodo (for example, fluoro (F) and chloro (Cl), such as F).

Unless otherwise specified, C_{1-z} alkyl groups (where z is the upper limit of the range) defined herein may be straight-chain or, when there is a sufficient number (i.e. a minimum of three) of carbon atoms, be branched-chain and/or cyclic (so forming a C_{3-z}-cycloalkyl group). When there is a sufficient number (i.e. a minimum of four) of carbon atoms, such groups may also be part cyclic. Part cyclic alkyl groups that may be mentioned include cyclopropylmethyl and cyclohexylethyl. When there is a sufficient number of carbon atoms, such groups may also be multicyclic (e.g. bicyclic or tricyclic) or spirocyclic.

For the avoidance of doubt, alkyl groups may be linear (otherwise referred to as straight-chained), branched (otherwise referred to as branched-chain) and/or cyclic. More particularly, alkyl groups may be linear (otherwise referred to as straight-chained) or branched (otherwise referred to as branched-chain).

Unless otherwise specified, C_{2-z} alkenyl groups (where z is the upper limit of the range) defined herein may be straight-chain or, when there is a sufficient number (i.e. a minimum of three) of carbon atoms, be branched-chain.

Unless otherwise specified, C_{2-z} alkynyl groups (where z is the upper limit of the range) defined herein may be straight-chain or, when there is a sufficient number (i.e. a minimum of four) of carbon atoms, be branched-chain.

For the avoidance of doubt, the skilled person will understand that the term alkyl will refer to saturated hydrocarbon moieties, whereas the term alkenyl will refer to unsaturated hydrocarbon moieties containing at least one carbon-carbon double bond and the term alkynyl will refer to unsaturated hydrocarbon moieties containing at least one carbon-carbon triple bond.

The skilled person will understand that that where the ring comprising Q¹ to Q⁵ (which may be referred to as ring Q) is heteroaryl, the ring will comprise, in addition to carbon atoms, one or more heteroatom, so as to form suitable heteroaryl groups as known to those skilled in the art. Moreover, the skilled person will understand that where the ring containing Q¹ to Q⁵ is 5-membered, one of Q¹ to Q⁵ (e.g. Q⁵) will represent a direct bond (i.e. that group will not be present).

For the avoidance of doubt, the depiction of the ring containing the Q¹ to Q⁵ groups with a circle therein (for example, as in formula I) will be understood to indicate that the ring is aromatic.

Various heteroaryl groups will be well-known to those skilled in the art, such as pyridinyl, pyridonyl, pyrrolyl, furanyl, thiophenyl, oxadiazolyl, thiadiazolyl, thiazolyl, oxazolyl, pyrazolyl, triazolyl, tetrazolyl, isoxazolyl, isothiazolyl, imidazolyl and the like. The oxides of heteroaryl/heteroaromatic groups are also embraced within the scope of the invention (e.g. the N-oxide).

In particular, the term heteroaryl (or heteroaromatic) includes references to 5-membered or 6-membered heteroaromatic groups containing at least one N atom and optionally one additional heteroatoms selected (e.g. from oxygen, nitrogen and/or sulfur). Particular heteroaryl groups that may be mentioned include those comprising, in the heteroaryl ring, at least one N atom (e.g. one N atom).

Particular heteroaryl groups (e.g. representing ring Q) that may be mentioned include thiazolyl (e.g. thiazol-4-yl and thiazol-5-yl, also thiazol-2-yl), pyrazinyl, pyridazinyl (e.g. pyridazin-3-yl or pyridazin-4-yl, pyrimidinyl (e.g. pyrimidin-4-yl or pyrimidin-5-yl) and pyridonyl (e.g. pyridon-4-yl or pyridon-5-yl). For the avoidance of doubt, the skilled person will understand that pyridonyl groups may exist as the aromatic tautomers thereof, i.e. as hydroxy pyridinyl groups.

In particular embodiments, heteroaryl groups (e.g. representing ring Q) may be 6-membered heteroaromatic groups containing one or more (e.g. one) N atom.

In particular, heteroaryl groups (e.g. representing ring Q) that may be mentioned include pyridin-3-yl and pyridin-4-yl.

As will be understood by those skilled in the art, substituents on heteroaryl groups (e.g. groups representing X¹) may, as appropriate, be located on any atom in the ring system, including a heteroatom (i.e. a N atom). In such circumstances, the skilled person will understand that reference to the substituent being present “as appropriate” will indicate that certain substituents may only be present in positions wherein the presence of such a substituent is chemically allowable, as understood by those skilled in the art.

For the avoidance of doubt, the skilled person will understand that the identities of Q¹ to Q⁵ will be selected such that the resulting heteroaryl is a suitable heteroaryl as known to those skilled in the art. For example, in certain instances, where the Q containing ring is a 5-membered ring, one of Q¹ to Q⁵ will represent a direct bond.

Similarly, the skilled person will understand that in 5-membered heteroaryl rings there can only be one O or S, but up to four N atoms (with up to four heteroatoms in total), which may be substituted as appropriate. Similarly, in a 6-membered heteroaryl ring there will be no O or S present in the ring but up to four (e.g. one or two) N atoms, which will be present as N.

For the avoidance of doubt, as used herein, references to heteroatoms will take their normal meaning as understood by one skilled in the art. Particular heteroatoms that may be mentioned include phosphorus, selenium, tellurium, silicon, boron, oxygen, nitrogen and sulphur (in particular, oxygen, nitrogen and sulphur).

The skilled person will understand that that where the ring comprising Q¹ to Q⁵ is aryl, the ring may represent in particular phenyl or naphthyl (particularly phenyl).

For the avoidance of doubt, references to polycyclic (e.g. bicyclic or tricyclic) groups (e.g. when employed in the context of cycloalkyl groups) will refer to ring systems wherein at least two scissions would be required to convert such rings into a straight chain, with the minimum number of such scissions corresponding to the number of rings defined (e.g. the term bicyclic may indicate that a minimum of two scissions would be required to convert the rings into a straight chain). For the avoidance of doubt, the term bicyclic (e.g. when employed in the context of alkyl groups) may refer to groups in which the second ring of a two-ring system is formed between two adjacent atoms of the first ring, and may also refer to groups in which two non-adjacent atoms are linked by an alkylene group, which later groups may be referred to as bridged.

For the avoidance of doubt, in cases in which the identity of two or more substituents in a compound of the invention may be the same, the actual identities of the respective substituents are not in any way interdependent. For example, in the situation in which two or more X¹ groups are present, those X¹ groups may be the same or different. Similarly, where two or more X¹ groups are present and each represent halo, the halo groups in question may be the same or different.

The skilled person will appreciate that compounds of the invention that are the subject of this invention include those that are stable. That is, compounds of the invention include those that are sufficiently robust to survive isolation, e.g. from a reaction mixture, to a useful degree of purity.

All embodiments of the invention and particular features mentioned herein may be taken in isolation or in combination with any other embodiments and/or particular features mentioned herein (hence describing more particular embodiments and particular features as disclosed herein) without departing from the disclosure of the invention.

In certain embodiments of the first aspect of the invention, RX¹ represents linear or branched (e.g. linear) C₁₋₁₂ alkyl, such as linear or branched (e.g. linear) C₁₋₆ or C₁₋₃ alkyl, optionally substituted by one G³ (i.e. a maximum of one G³ group).

In particular embodiments, R¹ represents a C₁₋₃ alkyl , such as a linear C₁₋₃ alkyl, optionally substituted by one G³.

For example, RX¹ may represent methyl or propyl (e.g. n-propyl), such as methyl, optionally substituted by one G³.

In particular embodiments, R¹ is not substituted (i.e. there is no G³ group present).

In particular embodiments, R¹ is substituted by one G³ (i.e. there is one G³ group present).

In particular embodiments, R^{h1} represents

C₁₋₆ alkyl , or

phenyl optionally substituted by one or more (e.g. up to three, such as one or two, particularly one) halo (e.g. F or Cl), such as 4-Cl-phenyl.

For example, in certain embodiments R^{h1} represents

C₁₋₆ alkyl (e.g. C₁₋₃ alkyl, such as methyl), or

phenyl optionally substituted by one or more (e.g. one) halo (e.g. F or Cl, such as Cl), such as 4-Cl-phenyl.

In particular embodiments that may be mentioned, Rhl represents methyl, or

phenyl optionally substituted with one or more (e.g. one) halo (e.g. Cl), such as 4-Cl-phenyl.

In certain embodiments, the compound of formula I is a compound of formula IA

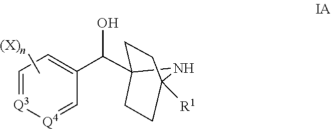

(i.e. the compounds of the invention are a compound of formula IA, or a pharmaceutically acceptable salt thereof), wherein:

Q³ and Q⁴ each represent N or C;

when Q³ and Q⁴ both represent C, each X independently represents X¹;

when one or more of Q³ and Q⁴ represent N, each X independently represents X²;

wherein n represents 0 to 5, as appropriate (e.g. wherein n represents 0 to 3); and

R¹, X¹ and X² are as defined herein.

In particular embodiments, either Q³ and Q⁴ each C (e.g. CH), or one of Q³ and Q⁴ represents N and the other represents C (e.g. CH).

For the avoidance of doubt, the skilled person will note that where Q⁴ represents C, unless that position is substituted by X¹ the relevant C will be present as a CH moiety.

In certain embodiments, Q³ represents N and Q⁴ represents C (particularly wherein the heteroaryl ring Q is 6-membered, e.g. such that ring Q is pyridin-4-yl).

In certain embodiments, Q⁴ represents N and Q³ represents C (particularly wherein the heteroaryl ring Q is 6-membered, e.g. such that ring Q is pyridin-3-yl).

In certain embodiments, Q³ and Q⁴ each represent C (e.g. such that ring Q is phenyl).

In certain embodiments, X¹ and X² each independently represent halo, OH, CN, C₁₋₆ alkyl or NH₂.

In particular embodiments, X¹ and X² each independently represent halo (e.g. F or Cl).

For example, in a particular embodiment each X¹ independently represents F or Cl, and each X² represents F.

In certain embodiments, n represents 0 to 2 (such as 0 or 1). In particular embodiments, n represents 1.

In certain embodiments of the first aspect of the invention, the compound of formula I is a compound of formula IB

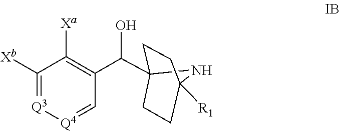

(i.e. the compound of the invention is a compound of formula IB, or a pharmaceutically acceptable salt thereof), wherein:

when at least one (e.g. one) of Q³ or Q⁴ represents N, X^{a} and X^{b} each represents H or X²;

when Q³ and Q⁴ both represent C, then X^{a} and X^{b} each represents H or X¹;

X¹, X², Q³, Q⁴ and R¹ are as defined herein.

In certain embodiments, X^{a} represents H and X^{b} represents X¹ or X², as appropriate.

In certain embodiments, X^{a} represents X¹ or X², as appropriate, and X^{b} represents H.

In certain embodiments of the first aspect of the invention, the compound of formula is a compound of formula IC

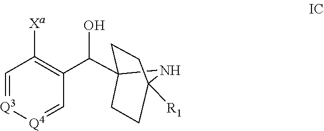

wherein:
X^{a}, Q³, Q⁴ and R¹ are as defined herein.

In certain embodiments of the first aspect of the invention, the compound of formula I is a compound of formula ID

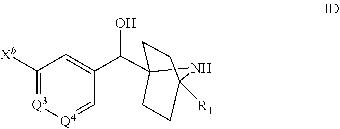

wherein:

X^{b}, Q³, Q⁴ and R¹ are as defined herein.

In certain embodiments, where Q³ represents N and Q⁴ represents CH, X^{a} represents F and X^{b} represents H.

In certain embodiments, where Q³ represents CH and Q⁴ represents N, X^{a} represents H and X^{b} represents F.

In certain embodiments of the first aspect of the invention, the compound of formula I is a compound of formula IE

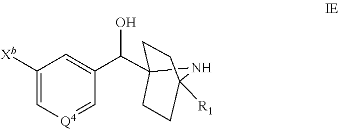

wherein:
Q⁴ represents CH or N, and R¹ and X^{b} are defined herein; in particular, wherein X^{b} represents Cl or F.

As described herein, compounds of the first aspect of the invention may also contain one or more asymmetric carbon atoms and may therefore exhibit optical and/or diastereoisomerism. Moreover, it has been found that certain such optical and/or diastereoisomers may show increased utility in the treatment of hyperglycaemia or disorders characterized by hyperglycaemia (such as type 2 diabetes), as described herein.

In a certain embodiment of the first aspect of the invention, the right-hand side of the compound may be referred to by the following structure

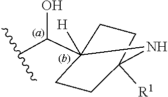

The skilled person will understand that that the carbon substituted with the essential —OH group (i.e. carbon (a)) is chiral and may be in either the (R) or (S) configuration. Likewise, the carbon beta to the hydroxy group and adjoined to the ring system (i.e. carbon (b)) is chiral and may be in either the (R) or (S) configuration.

In a particular embodiment, carbon (a) is in the (R) configuration and carbon (b) is in the (S) configuration.

In a particular embodiment, carbon (a) is in the (S) configuration and carbon (b) is in the (R) configuration.

In a particular embodiment, carbon (a) is in the (R) configuration and carbon (b) is in the (R) configuration.

In a particular embodiment, carbon (a) is in the (S) configuration and carbon (b) is in the (S) configuration.

In particular embodiments, where one stereocentre (i.e. carbon (a) or carbon (b)) has a specified stereochemistry, the compound will be present in the substantial absence of the other (opposite) stereoisomer.

In particular embodiments, where two stereocentres (i.e. carbon (a) and carbon (b)) have a specified stereochemistry, the compound will be present in the substantial absence of the other diastereoisomers.

As used herein, references to the substantial absence of other stereoisomers will refer to the desired stereoisomer being present at a purity of at least 80% (e.g. at least 90%, such as at least 95%) relative to the other stereoisomers. Alternatively, the relevant stereochemical configuration may be referred to as being present in an enantiomeric excess (e.e.) or diastereomeric excess (d.e.), as appropriate, of at least 90% (such as at least 95%, at least 98% or, particularly, at least 99%, for example at 35 least 99.9%).

For the avoidance of doubt, compounds referred to as having a specific stereochemistry at a defined position (e.g. in the case of compounds of formula I, the carbon (a) in the (R) or (S) configuration) may also have stereochemistry at one or more other positions, and so may exist as mixtures of enantiomers or diastereoisomers in relation to the stereochemistry at those positions.

Particular compounds of the invention (including compounds of formula I and all embodiments and particular forms thereof) that may be mentioned include the compounds of the examples as provided herein, or a pharmaceutically acceptable salt thereof.

Where an example compound is indicated to have been obtained in a particular salt form, the skilled person will understand that particular compounds of the invention that may be mentioned include the free base or free acid (as appropriate) of that compound, and vice versa. Further, where an example compound is indicated to have been obtained in a particular salt form, particular compounds of the invention that may be mentioned include other (i.e. different) pharmaceutically acceptable salts of that compound.

Medical Uses

As indicated herein, the compounds of the invention, and therefore compositions and kits comprising the same, are useful as pharmaceuticals.

Thus, according to a second aspect of the invention there is provided a compound of the first aspect of the invention, as hereinbefore defined (i.e. a compound as defined in the first aspect of the invention, including all embodiments and particular features thereof), for use in medicine (i.e. for use as a pharmaceutical, which may be described as use as a medicament).

Compounds described herein are β₂ adrenergic receptor agonists and therefore suitable in treating diseases such as those described herein. Such activity may be observed in compounds of the invention by identifying compounds which stimulate the uptake of glucose in skeletal muscle cells, which activity may be confirmed to be mediated by activation of the β₂ receptor by observation that such activity is prevented or diminished in the presence of a (e.g. selective) β₂ adrenergic receptor antagonist (such as in the biological examples provided herein).

Thus, in a third aspect of the invention, there is provided a compound of the first aspect of the invention, as hereinbefore defined, for use in treating a disease or disorder the treatment of which is mediated by activation of the β₂ adrenergic receptor.

In an alternative third aspect of the invention, there is provided the use of a compound of the first aspect of the invention in the manufacture of a medicament for use in treating a disease or disorder the treatment of which is mediated by activation of the β₂ adrenergic receptor.

In a further alternative third aspect of the invention, there is provided a method of treating a disease or disorder the treatment of which is mediated by activation of the β₂ adrenergic receptor comprising administering to a patient in need thereof a therapeutically effective amount of a compound of the first aspect of the invention.

For the avoidance of doubt, references to compounds as defined in the first aspect of the invention will include references to compounds of formula I (including all embodiments thereof) and pharmaceutically acceptable salts thereof.

As indicated herein, the compounds of the invention act by inducing uptake of glucose in skeletal muscle cells, thus allowing for the reduction of blood glucose levels in vivo. Thus, compounds of the invention may be of particular use in treating hyperglycaemia or a disorder characterized by hyperglycaemia.

In a particular embodiment of the third aspect of the invention, there is provided a compound of the first aspect of the invention, as hereinbefore defined, for use in the treatment of hyperglycaemia or a disorder characterized by hyperglycaemia.

In an alternative embodiment of the third aspect of the invention, there is provided the use of a compound of the first aspect of the invention in the manufacture of a medicament for use in the treatment of hyperglycaemia or a disorder characterized by hyperglycaemia.

In a further alternative embodiment of the third aspect of the invention, there is provided a method of treating hyperglycaemia or a disorder characterized by hyperglycaemia comprising administering to a patient in need thereof a therapeutically effective amount of a compound of the first aspect of the invention.

For the avoidance of doubt, the term “hyperglycaemia” as used herein will be understood by those skilled in the art to refer to a condition wherein an excessive amount of glucose circulates in blood plasma of the subject experiencing the same. In particular, it may refer to a subject (e.g a human subject) having blood glucose levels higher than about 10.0 mmol/L (such as higher than about 11.1 mmol/L, e.g. higher than about 15 mmol/L), although it may also refer to a subject (e.g. a human subject) having blood glucose levels higher than about 7 mmol/L for an extended period of time (e.g. for greater than 24 hours, such as for greater than 48 hours).

The skilled person will understand that references to the treatment of a particular condition (or, similarly, to treating that condition) take their normal meanings in the field of medicine. In particular, the terms may refer to achieving a reduction in the severity of one or more clinical symptom associated with the condition. For example, in the case of type 2 diabetes, the term may refer to achieving a reduction of blood glucose levels. In particular embodiments, in the case of treating hyperglycaemia or conditions characterised by hyperglycaemia, the term may refer to achieving a reduction of blood glucose levels (for example, to or below about 10.0 mmol/mL (e.g. to levels in the range of from about 4.0 mmol/L to about 10.0 mmol/L), such as to or below about 7.5 mmol/mL (e.g. to levels in the range of from about 4.0 mmol/L to about 7.5 mmol/L) or to or below about 6 mmol/mL (e.g. to levels in the range of from about 4.0 mmol/L to about 6.0 mmol/L)).

As used herein, references to patients will refer to a living subject being treated, including mammalian (e.g. human) patients. Thus, in particular embodiments of the first aspect of the invention, the treatment is in a mammal (e.g. a human).

As used herein, the term therapeutically effective amount will refer to an amount of a compound that confers a therapeutic effect on the treated patient. The effect may be objective (i.e. measurable by some test or marker) or subjective (i.e. the subject gives an indication of and/or feels an effect).

Although compounds of the first aspect of the invention may possess pharmacological activity as such, certain pharmaceutically-acceptable (e.g. “protected”) derivatives of compounds of the invention may exist or be prepared which may not possess such activity, but may be administered parenterally or orally and thereafter be metabolised in the body to form compounds of the invention. Such compounds (which may possess some pharmacological activity, provided that such activity is appreciably lower than that of the active compounds to which they are metabolised) may therefore be described as “prodrugs” of compounds of the invention.

As used herein, references to prodrugs will include compounds that form a compound of the invention, in an experimentally-detectable amount, within a predetermined time, following enteral or parenteral administration (e.g. oral or parenteral administration). All prodrugs of the compounds of the first aspect of the invention are included within the scope of the invention.

For the avoidance of doubt, the compounds of the first aspect of the invention are useful because they possess pharmacological activity, and/or are metabolised in the body following oral or parenteral administration to form compounds that possess pharmacological activity. In particular, as described herein, compounds of the first aspect of the invention are useful in the treatment of hyperglycaemia or disorders characterized by hyperglycaemia (such as type 2 diabetes), which terms will be readily understood by one of skill in the art (as described herein).

In a particular embodiment, the treatment is of a disorder (which may also be referred to as a condition or disease) characterised by hyperglycaemia.

In particular embodiments, compounds of the invention (i.e. compounds of formula I, including all embodiments thereof) are for use in the treatment of type 2 diabetes (or useful in the manufacture of a medicament for such treatment, or useful in a method for such treatment, as described herein).

In particular embodiments of the first aspect of the invention, the disorder is type 2 diabetes, such as type 2 diabetes of a sub-type selected from the list consisting of maturity-onset diabetes in the young (MODY), ketosis-prone diabetes in adults, latent autoimmune diabetes of adults (LADA), and gestational diabetes.

In further particular embodiments, the treatment of type 2 diabetes is in a non-obese patient.

For the avoidance of doubt, the skilled person will understand that patients with a Body Mass Index (BMI) of greater than 30 are considered to be obese.

In particular embodiments, the treatment may be of hyperglycaemia in a patent who is at risk of developing type 2 diabetes, which condition may be defined as pre-diabetes. Thus, compounds of the invention may be useful in the prevention of type 2 diabetes (e.g. in a patient having pre-diabetes).

As used herein, the term prevention (and, similarly, preventing) includes references to the prophylaxis of the disease or disorder (and vice-versa). As such, references to prevention may also be references to prophylaxis, and vice versa. In particular, the term may refer to achieving a reduction in the likelihood of the patient (or healthy subject) developing the condition (for example, at least a 10% reduction, such as at least a 20%, 30% or 40% reduction, e.g. at least a 50% reduction).

In more particular embodiments, the type 2 diabetes is characterised by the patient displaying severe insulin resistance (SIR).

In further embodiments, the treatment may be of hyperglycaemia in a patient having type X¹ diabetes. Thus, compounds of the invention may be useful in the treatment of hyperglycaemia in type X¹ diabetes.

The skilled person will understand that compounds of the invention may be useful in treating hyperglycaemia in patients having impaired insulin production, such as in patients having cystic fibrosis. Thus, in further embodiments, the disorder characterized by hyperglycaemia is cystic fibrosis-related diabetes.

In particular embodiments that may be mentioned, the disorder characterised by hyperglycaemia is (or is characterized by) severe insulin resistance (SIR), which may be understood by those in the art to refer to disorders wherein typically the subject has normal, or in some cases increased, insulin production but significantly reduced insulin sensitivity. In particular instances, such patients may be non-obese (e.g. being of a healthy weight). Thus, in particular embodiments, such treatments are performed in patients who are not defined as being obese (e.g. in patients who are defined as being of a healthy weight).

For example, SIR may be identified in a patient based in said patient having fasting insulin>150 pmol/L and/or a peak insulin on glucose tolerance testing of >1,500 pmol/L, particularly in individuals with a BMI<30 kg/m² (which patient may otherwise have normal glucose tolerance).

More particularly, SIR may be characterised by the patient having no significant response to the presence of insulin, which may result from a defect (e.g. a genetic defect) in the function of the insulin receptor.

Particular disorders that may be characterised by SIR include: Rabson-Mendenhall syndrome, Donohue's syndrome (leprechaunism), Type A and Type B syndromes of insulin resistance, the HAIR-AN (hyperandrogenism, insulin resistance, and acanthosis nigricans) syndromes, pseudoacromegaly, and lipodystrophy.

More particular disorders that may be characterised by SIR include Donohue's syndrome and Type A syndrome of insulin resistance and, yet more particularly, Rabson-Mendenhall syndrome.

The skilled person will understand that treatment with compounds of the first aspect of the invention may further comprise (i.e. be combined with) further (i.e. additional/other) treatment(s) for the same condition. In particular, treatment with compounds of the invention may be combined with other means for the treatment of type 2 diabetes, such as treatment with one or more other therapeutic agent that is useful in the treatment of type 2 diabetes as known to those skilled in the art, such as therapies comprising requiring the patient to undergo a change of diet and/or undertake exercise regiments, and/or surgical procedures designed to promote weight loss (such as gastric band surgery).

In particular, treatment with compounds of the invention may be performed in combination with (e.g. in a patient who is also being treated with) one or more (e.g. one) additional compounds (i.e. therapeutic agents) that:
(i) are capable of reducing blood sugar levels; and/or
(ii) are insulin sensitizers; and/or
(iii) enhance insulin release,
   all of which are described herein below.

In alternative embodiments, compounds of the first aspect of the invention (i.e. compounds of the invention) may be useful in the treatment of a non-alcoholic fatty liver disease (NAFLD).

Non-alcoholic fatty liver disease (NAFLD) is defined by excessive fat accumulation in the form of triglycerides (steatosis) in the liver (designated as an accumulation of greater than 5% of hepatocytes histologically). It is the most common liver disorder in developed countries (for example, affecting around 30% of US adults) and most patients are asymptomatic. If left untreated, the condition may progressively worsen and may ultimately lead to cirrhosis of the liver. NAFLD is particularly prevalent in obese patents, with around 80% thought to have the disease.

A sub-group of NAFLD patients (for example, between 2 and 5% of US adults) exhibit liver cell injury and inflammation in addition to excessive fat accumulation. This condition, designated as non-alcoholic steatohepatitis (NASH), is virtually indistinguishable histologically from alcoholic steatohepatitis. While the simple steatosis seen in NAFLD does not directly correlate with increased short-term morbidity or mortality, progression of this condition to NASH dramatically increases the risks of cirrhosis, liver failure and hepatocellular carcinoma. Indeed, NASH is now considered to be one of the main causes of cirrhosis (includeing cryptogenic cirrhosis) in the developed world.

The exact cause of NASH has yet to be elucidated, and it is almost certainly not the same in every patient. It is most closely related to insulin resistance, obesity, and the metabolic syndrome (which includes diseases related to diabetes mellitus type 2, insulin resistance, central (truncal) obesity, hyperlipidaemia, low high-density lipoprotein (HDL) cholesterol, hypertriglyceridemia, and hypertension). However, not all patients with these conditions have NASH, and not all patients with NASH suffer from one of these conditions. Nevertheless, given that NASH is a potentially fatal condition, leading to cirrhosis, liver failure and hepatocellular carcinoma, there exists a clear need for an effective treatment.

In particular embodiments, compounds of the invention (i.e. compounds of formula I, including all embodiments thereof) are for use in the treatment of a non-alcoholic fatty liver disease (or useful in the manufacture of a medicament for such treatment, or useful in a method for such treatment, as described herein).

The process by which the triglyceride fat accumulates in liver cells is called steatosis (i.e. hepatic steatosis). The skilled person will understand that the term “steatosis” encompasses the abnormal retention of fat (i.e. lipids) within a cell. Thus, in particular embodiments of the first aspect of the invention, the treatment or prevention is of a fatty liver disease which is characterized by steatosis.

During steatosis, excess lipids accumulate in vesicles that displace the cytoplasm of the cell. Over time, the vesicles can grow large enough to distort the nucleus, and the condition is known as macrovesicular steatosis. Otherwise, the condition may be referred to as microvesicular steatosis. Steatosis is largely harmless in mild cases; however, large accumulations of fat in the liver can cause significant health issues. Risk factors associated with steatosis include diabetes mellitus, protein malnutrition, hypertension, obesity, anoxia, sleep apnea and the presence of toxins within the cell.

As described herein, fatty liver disease is most commonly associated with alcohol or a metabolic syndrome (for example, diabetes, hypertension, obesity or dyslipidemia). Therefore, depending on the underlying cause, fatty liver disease may be diagnosed as alcohol-related fatty liver disease or non-alcoholic fatty liver disease (NAFLD).

Particular diseases or conditions that are associated with fatty liver disease that are not related to alcohol include metabolic conditions such as diabetes, hypertension, obesity, dyslipidemia, abetalipoproteinemia, glycogen storage diseases, Weber-Christian disease, acute fatty liver of pregnancy, and lipodystrophy. Other non-alcohol related factors related to fatty liver diseases include malnutrition, total parenteral nutrition, severe weight loss, refeeding syndrome, jejunoileal bypass, gastric bypass, polycystic ovary syndrome and diverticulosis.

The compounds of the invention have been found to be particularly useful in the treatment or prevention of NAFLD, which may be referred to as a fatty liver disease which is not alcohol related. A fatty liver disease which is “not alcohol related” may be diagnosed wherein alcohol consumption of the patient is not considered to be a main causative factor. A typical threshold for diagnosing a fatty liver disease as “not alcohol related” is a daily consumption of less than 20 g for female subjects and less than 30 g for male subjects.

If left untreated, subjects suffering from fatty liver disease may begin to experience inflammation of the the liver (hepatitis). It has been postulated that one of the possible causes of this inflammation may be lipid peroxidative damage to the membranes of the liver cells. Inflammation of a fatty liver can lead to a number of serious conditions and it is therefore desirable to treat or prevent fatty liver disease before inflammation occurs. Thus, in particular embodiments of the first aspect of the invention, the treatment or prevention is of a NAFLD which is associated with inflammation.

Non-alcoholic steatohepatitis (NASH) is the most aggressive form of NAFLD, and is a condition in which excessive fat accumulation (steatosis) is accompanied by inflammation of the liver. If advanced, NASH can lead to the development of scar tissue in the liver (fibrosis) and, eventivally, cirrhosis. As described above, the compounds of the invention have been found to be useful in the treatment or prevention of NAFLD, particularly when accompanied by inflamation of the liver. It follows that the compounds of the invention are also useful in the treatment or prevention of NASH. Therefore, in a further embodiment of the first aspect of the invention, the treatment or prevention is of non-alcoholic steatohepatitis (NASH).

The skilled person will understand that treatment with compounds of the first aspect of the invention may further comprise (i.e. be combined with) further (i.e. additional/other) treatment(s) for the same condition. In particular, treatment with compounds of the invention may be combined with other means for the treatment of a fatty liver disease, as described herein, such as treatment with one or more other therapeutic agent that is useful in the treatment of a fatty liver disease as known to those skilled in the art; for example, therapies comprising requiring the patient to undergo a change of diet and/or undertake exercise regiments, and/or surgical procedures designed to promote weight loss (such as gastric band surgery).

In particular, treatment with compounds of the invention may be performed in combination with (e.g. in a patient who is also being treated with) one or more (e.g. one) additional compounds (i.e. therapeutic agents) that are capable of reducing the level of fat (e.g. triglycerides) in the liver.

References to treatment of a fatty liver disease may refer to achieving a therapeutically significant reduction of fat (e.g. triglycerides levels) in liver cells (such as a reduction of at least 5% by weight, e.g. a reduction of at least 10%, or at least 20% or even 25%).

As described herein, compounds of the invention may be of use in treating a disease or disorder the treatment of which is mediated by activation of the β₂ adrenergic receptor.

In particular embodiments, the compounds of the first aspect of the invention may be understood to positively modulate the β₂ adrenergic receptor, which compounds may be referred to as a β₂-adrenergic receptor agonist.

The skilled person will appreciate what is meant by “β₂ adrenergic receptor” (or “β₂-AR”). Such receptors are known in the art and have been reviewed in, e.g., Johnson. M., J. Allergy Clin. Immunol., 117, 18-24 (2006). For the avoidance of doubt, adrenergic receptors are a class of G protein-coupled receptors which bind and are activated by their endogenous ligands, the catecholamines, adrenaline and noradrenaline. The adrenergic receptor falls into five types: α₁, α₂, β₁, β₂ and β₃. These subtypes are expressed in distinct patterns and involved in different physiological processes, such that ligands that can selectively target one subtype have therapeutic potential for multiple diseases. The present invention is concerned with the β₂ adrenergic receptor, although compounds may interact with one or more other adrenergic receptor (e.g. one or more other β adrenergic receptor).

The term “positively modulates β₂-adrenergic receptor activity” will be understood to mean that the compound is capable of altering the signalling of the receptor.

As used herein, the term “β₂ agonist” is used to mean β₂ adrenergic receptor agonist. In certain embodiments, the term β₂ agonist is understood to include compounds that are primarily β₂ agonists, but may also exhibit some agonism for other adrenergic receptors. In this application, the terms “β₂ adrenergic receptor agonist”, “β₂ AR agonist”, “β₂AR agonist” and “β₂ agonist” may be used interchangeably.

Thus, in certain embodiments, references to β₂ agonists may include both selective and non-selective agonists.

In certain embodiments, references to β₂ agonists may include any ligand that change receptor signalling including but not limited to full and partial agonists. Further, β₂ agonists that may be used in accordance with various aspects and embodiments of the present disclosure may be short-acting, long acting or ultra long-acting.

As used herein, the term “mediated by activation of the β₂ adrenergic receptor” is used to indicate that activation of the receptor regulates or causes a physiological response which will in turn provide a biological effect corresponding to (or leading to) treatment of the disease or disorder.

As used in herein, references to diseases and disorders the treatment of which is “mediated by activation of the β₂ adrenergic receptor” may also refer to diseases and disorders (and in particular the treatment thereof) being, inter alfa, “associated with”, “mediated by”, “affected by”, “regulated by”, “modulated by” and “linked to” the β₂ adrenergic receptor.

As described herein, diseases and disorders the treatment of which is mediated by activation of the β₂ adrenergic receptor will be known to those skilled in the art. Thus, the skilled person will understand that in respect of certain of the diseases and disorders described herein the suitability of compounds of the invention for the treatment of such diseases and disorders may be known to those skilled in the art; for example, based on the disclosures referred to herein below (the contents of which are incorporated herein by reference).

In addition to those as may be described herein above, particular diseases and disorders the treatment of which is mediated by activation of the β₂ adrenergic receptor that may be mentioned include:
neurodegenerative diseases, such as MCI (mild cognitive impairment), aMCI (amnestic MCI), vascular dementia, mixed dementia, FTD (front-temporal dementia), HD (Huntington disease), Rett syndrome, PSP (progressive supranuclear palsy), CBD (corticobasal degeneration), SCA (spinocerebellar ataxia), MSA (multiple system atrophy), SDS (Shy-Drager syndrome), olivopontocerebellar atrophy, TBI (traumatic brain injury), CTE (chronic traumatic encephalopathy), stroke, EKS (Wernicke-Korsakoff syndrome), normal pressure hydrocephalus, hypersomnia (narcolepsy), ASD (autistic spectrum disorders), FXS (fragile X syndrome), YSC (tubular sclerosis complex), prion-related disorders, CJD (Creutzfeldt-Jakob disease), depressive disorders, DLC (dementia with Lewy bodies), PD (Parkinson's disease), PDD (PD dementia), ADHD (attention deficit hyperactivity disorder), Alzheimer's disease (AD), early AD and DS (Down syndrome);
muscle dystrophy or a disorder characterised by muscular dystrophy, such as muscle damage, muscle wasting, muscle atrophy, muscle degeneration or sclerosis;
kidney disease, such as CKD (chronic kidney disease), ESRD (end-stage renal disease) and diabetic nephropathy;
inflammation or a disorder characterised by inflammation, such as sepsis, psoriasis, dermatitis, psoriasis-like skin dermatitis, lacerations or HDF (human dermal fibroblasts), and including localised acute inflammation, such as that related to endotoxemia and Acute Lung Injury (ALI), and respiratory conditions associated with inflammation, such as asthma and other pulmonary disorders, such as chronic obstructive pulmonary disease (COPD); and
an autoimmune disease, such as SLE (systemic lupus erythematosus, RA (rheumatoid arthritis), MG (myasthenia gravis) MS and GD (Grave's disease).

The suitability of β₂ adrenergic receptor agonists for treating such conditions may be demonstrated by the data provided herein and by reference to the literature known to those skilled on the art, such as that described herein (the whole contents of which, in particular the experimental results presented, will be understood to be incorporated herein by reference).

In particular, the suitability of β₂ adrenergic receptor agonists for treating certain of the diseases and disorders referred to herein may be identified in and, in some instances, confirmed by the disclosures of WO 2020/198466 A1 and WO 2021/003161 A1 (which, for the avoidance of doubt, are incorporated herein by reference, in particular the examples as provided therein).

In a particular embodiment, there is provided a compound of the first aspect of the invention, as hereinbefore defined, for use in treating neurodegenerative diseases.

In particular embodiments, the neurodegenerative disease is selected from MCI (mild cognitive impairment), aMCI (amnestic MCI), vascular dementia, mixed dementia, FTD (front-temporal dementia), HD (Huntington disease), Rett syndrome, PSP (progressive supranuclear palsy), CBD (corticobasal degeneration), SCA (spinocerebellar ataxia), MSA (multiple system atrophy), SDS (Shy-Drager syndrome), olivopontocerebellar atrophy, TBI (traumatic brain injury), CTE (chronic traumatic encephalopathy), stroke, EKS (Wernicke-Korsakoff syndrome), normal pressure hydrocephalus, hypersomnia (narcolepsy), ASD (autistic spectrum disorders), FXS (fragile X syndrome), YSC (tubular sclerosis complex), prion-related disorders, CJD (Creutzfeldt-Jakob disease), depressive disorders, DLC (dementia with Lewy bodies), PD (Parkinson's disease), PDD (PD dementia), ADHD (attention deficit hyperactivity disorder), Alzheimer's disease (AD), early AD and DS (Down syndrome).

Mittal. S., et al., Science., 357(6354), 891-898 (2017) describes that β₂-adrenergic receptor agonists promote dopamine neuron health by reducing SNCA expression through H2K27 deacetylation and mitochondrial free radicals. This may benefit nigral dopamine neurons, which are prone to mitochondrial bioenergetics dysfunction at early stages of Lewy body neuropathy. β₂-adrenergic receptor agonists are expressed in the substantia nigra and cortex, regions that are progressively affected by Parkinson's disease (PD). Therefore, β₂-adrenergic receptor agonists can be used to reduce the risk and affect of PD.

Hishida. R., The Lancet, 870 (1992) describes that β₂-adrenergic receptor agonists can beneficially affect wearing-off in patients with Parkinson's disease on long-term levodopa.

UC, E. Y., et al., Clin. Neuropharmacol., 26(4), 207-212 (2003) describes that β₂-adrenergic receptor agonist, albuterol, benefited patients with PD through two mechanisms, an increased response to levodopa and an increase in muscle mass.

O'Neill, et al., Br. J. Pharmacol., 177, 282-297 (2019) describes that β₂-adrenergic receptor agonists restrict microglial activation and protect against the onset and progression of dopamine neuronal cell loss and related motor deficits provoke by central or systemic inflammation. Therefore, targeting β₂-adrenergic receptors with a β₂-adrenergic receptor agonist imbues an intervening prophylactic mechanism to protect against the progression of neurodegeneration and exacerbated decline in motor function associated with systemic and central inflammation. As a result, β₂-adrenergic receptor agonists may be beneficial in the treatment of PD-related neuropathy and motor impairments induced by inflammation.

In alternative embodiments, there is provided a compound of the first aspect of the invention, as hereinbefore defined, for use in treating muscle dystrophy or a disorder characterised by muscular dystrophy.

In particular such embodiments, the muscle dystrophy is muscle damage, muscle wasting, muscle atrophy, muscle degeneration or sclerosis.

Jiang, G., et al., ISRN Pharma., 2011, 1-7 (2011) describes that β₂-AR agonists ameliorate animal wasting in denervation, amyotrophic lateral sclerosis, muscular dystrophy, disuse, aging and myocardial unloading models. Further, in patients with immobilization conditions or muscular dystrophy, β₂-AR agonists increase lean body mass and enhance skeletal muscle functions. Also, β₂-AR agonists were found to promote myocardial recovery in patients with myocardial unloading atrophy resulting from application of left ventricular assist devise.

Bartus, R. T., et al., Neurobiol. Dis., 85, 11-24, 2016 indicates that β₂-adrenergic receptor agonists may enhance muscle bulk and muscle strength in amyotrophic lateral sclerosis (ALS) patients by increasing neurotrophic factors.

In alternative embodiments, there is provided a compound of the first aspect of the invention, as hereinbefore defined, for use in treating kidney disease.

In particular such embodiments, the kidney disease is selected from CKD (chronic kidney disease), ESRD (end-stage renal disease) and diabetic nephropathy.

Cleveland, K., et al., FASEB Journal, 33(1), 514 (2019) describes that β₂-adrenergic receptor agonists have been shown to induce mitochondrial biogenesis (MB) and promote recovery from acute kidney injury, and may find use as a potential therapy for diabetic nephropathy (DN).

Jesinkey, S. R., et al., J. Am. Soc. Nephrol., 25, 1157-1162 (2014) describes the necessity for mitochondrial biogenesis as an adaptive response for meeting the increased metabolic and energy demands during organ recovery after an acute injury.

In particular, renal mitochondrial dysfunction has been linked to pathogenesis of acute kidney injury (AKI), a disorder characterised by a rapid decrease in kidney excretory function and subsequent retention of harmful waste products.

In alternative embodiments, there is provided a compound of the first aspect of the invention, as hereinbefore defined, for use in treating inflammation or a disorder characterised by inflammation.

In particular embodiments, the inflammation is (or is characterised by) sepsis, psoriasis, dermatitis, psoriasis-like skin dermatitis, lacerations or HDF (human dermal fibroblasts).

As the skilled person will know, inflammation is a tightly controlled process that ensures proper localization of immune cells, release of pro- and anti-inflammatory mediators, clearance of dead cells, and containment of the pathogen.

The skilled person will know that inflammation may also be a cause of respiratory conditions, such as asthma and other pulmonary disorders, such as chronic obstructive pulmonary disease (COPD).

Grailer, J. J. et al, J Innate Immun, 6, 607-618 (2014) shows that blockade of the β₂ adrenergic receptor reduced survival and enhanced injury in mouse models of endotoxemia and LPS-induced acute lung injury, respectively. These results demonstrate the suitability of β₂AR activation in the treatment of localised acute inflammation, such as that related to endotoxemia and Acute Lung Injury.

Agac, D., et al., Brain, Behaviour and Immunity, 74, 176-185 (2018) describes that there is a unique synergistic pathway that converts acute inflammatory signals into an anti-inflammatory response and likely explains a variety of phenomena known to be involved in β₂-adrenergic receptor agonists mediated immune suppression. In particular, β₂-adrenergic receptor agonists signalling directly controls anti-inflammatory cytokine, IL-10, expression. These results suggest the use of β₂AR agonists in the treatment of inflammatory disorders, such as sepsis.

Liu, F., et al., Cells, 511(9), 1-17 (2020) describes that β₂-adrenergic receptor agonists demonstrated significant anti-psoriasis effects, which may involve regulating the Th17/Tregs axis balances and glycerophospholipid metabolism in response to imiquimod (IMQ) induced psoriasis.

Provost, G. S., et al., J. Investig. Dermatol., 135, 279-288 (2015) describes that β₂-adrenergic receptor agonists reduces human dermal fibroblast (HDF) differentiation, therefore reducing scarring to a patient following a laceration or open wound.

In alternative embodiments, there is provided a compound of the first aspect of the invention, as hereinbefore defined, for use in treating an autoimmune disease.

In particular such embodiments, the autoimmune disease is selected from SLE (systemic lupus erythematosus, RA (rheumatoid arthritis), MG (myasthenia gravis) MS and GD (Grave's disease).

Wu, et al., Front. Pharmacol., 1313(9), 1-9 (2018) describes that β₂-adrenergic receptor agonists may be a target treatment for autoimmune diseases (AD), such as SLE (systemic lupus erythematosus, RA (rheumatoid arthritis), MG (mysasthenia gravis) MS and GD (Grave's disease).

Pharmaceutical Compositions

As described herein, compounds of the first aspect of the invention are useful as pharmaceuticals. Such compounds may be administered alone or may be administered by way of known pharmaceutical compositions/formulations.

In a fourth aspect of the invention, there is provided a pharmaceutical composition comprising a compound as defined in the first aspect of the invention (i.e. a compound of the invention), and optionally one or more pharmaceutically acceptable adjuvant, diluent and/or carrier.

The skilled person will understand that references herein to compounds of the first aspect of the invention being for particular uses (and, similarly, to uses and methods of use relating to compounds of the invention) may also apply to pharmaceutical compositions comprising compounds of the invention as described herein.

In a fifth aspect of the invention, there is provided a pharmaceutical composition for use in the treatment of hyperglycaemia or a disoder characterized by hyperglycaemia (as defined herein, such as type 2 diabetes) comprising a compound as defined in the first aspect of the invention, and optionally one or more pharmaceutically acceptable adjuvant, diluent and/or carrier.

In an alternative fifth aspect of the invention, there is provided a pharmaceutical composition for use in the treatment or prevention of a non-alcoholic fatty liver disease, as defined herein.

The skilled person will understand that compounds of the first (and, therefore, second and third) aspect of the invention may act systemically and/or locally (i.e. at a particular site).

The skilled person will understand that compounds and compositions as described in the first to fifth aspects of the invention will normally be administered orally, intravenously, subcutaneously, buccally, rectally, dermally, nasally, tracheally, bronchially, sublingually, intranasally, topically, by any other parenteral route or via inhalation, in a pharmaceutically acceptable dosage form. Pharmaceutical compositions as described herein will include compositions in the form of tablets, capsules or elixirs for oral administration, suppositories for rectal administration, sterile solutions or suspensions for parenteral or intramuscular administration, and the like. Alternatively, particularly where such compounds of the invention act locally, pharmaceutical compositions may be formulated for topical administration.

Thus, in particular embodiments of the fourth and fifth aspects of the invention, the pharmaceutical formulation is provided in a pharmaceutically acceptable dosage form, including tablets or capsules, liquid forms to be taken orally or by injection, suppositories, creams, gels, foams, inhalants (e.g. to be applied intranasally), or forms suitable for topical administration. For the avoidance of doubt, in such embodiments, compounds of the invention may be present as a solid (e.g. a solid dispersion), liquid (e.g. in solution) or in other forms, such as in the form of micelles.

For example, in the preparation of pharmaceutical formulations for oral administration, the compound may be mixed with solid, powdered ingredients such as lactose, saccharose, sorbitol, mannitol, starch, amylopectin, cellulose derivatives, gelatin, or another suitable ingredient, as well as with disintegrating agents and lubricating agents such as magnesium stearate, calcium stearate, sodium stearyl fumarate and polyethylene glycol waxes. The mixture may then be processed into granules or compressed into tablets.

Soft gelatin capsules may be prepared with capsules containing one or more active compounds (e.g. compounds of the first and, therefore, second and third aspects of the invention, and optionally additional therapeutic agents), together with, for example, vegetable oil, fat, or other suitable vehicle for soft gelatin capsules. Similarly, hard gelatine capsules may contain such compound(s) in combination with solid powdered ingredients such as lactose, saccharose, sorbitol, mannitol, potato starch, corn starch, amylopectin, cellulose derivatives or gelatin.

Dosage units for rectal administration may be prepared (i) in the form of suppositories which contain the compound(s) mixed with a neutral fat base; (ii) in the form of a gelatin rectal capsule which contains the active substance in a mixture with a vegetable oil, paraffin oil, or other suitable vehicle for gelatin rectal capsules; (iii) in the form of a ready-made micro enema; or (iv) in the form of a dry micro enema formulation to be reconstituted in a suitable solvent just prior to administration.

Liquid preparations for oral administration may be prepared in the form of syrups or suspensions, e.g. solutions or suspensions, containing the compound(s) and the remainder of the formulation consisting of sugar or sugar alcohols, and a mixture of ethanol, water, glycerol, propylene glycol and polyethylene glycol. If desired, such liquid preparations may contain colouring agents, flavouring agents, saccharine and carboxymethyl cellulose or other thickening agent. Liquid preparations for oral administration may also be prepared in the form of a dry powder to be reconstituted with a suitable solvent prior to use.

Solutions for parenteral administration may be prepared as a solution of the compound(s) in a pharmaceutically acceptable solvent. These solutions may also contain stabilizing ingredients and/or buffering ingredients and are dispensed into unit doses in the form of ampoules or vials. Solutions for parenteral administration may also be prepared as a dry preparation to be reconstituted with a suitable solvent extemporaneously before use.

The skilled person will understand that compounds of the invention, and pharmaceutically-acceptable salts thereof, may be administered (for example, as formulations as described hereinabove) at varying doses, with suitable doses being readily determined by one of skill in the art. Oral, pulmonary and topical dosages (and subcutaneous dosages, although these dosages may be relatively lower) may range from between about 0.01 μg/kg/day of body weight per day (μg/kg/day) to about 20 mg/kg/day of body weight per day (mg/kg/day), preferably about 0.1 μg/kg/day to about 5 mg/kg/day, and more preferably about X¹ μg/kg/day to about 2 mg/kg/day (e.g. about 10 μg/kg/day to about X¹ mg/kg/day). For example, when administered orally, treatment with such compounds may comprise administration of a formulations typically containing between about X¹ pg to about 2000 mg, for example between about 10 μg to about 500 mg, or between 100 μg to about 100 mg (e.g. about X¹ mg to about 100 mg), of the active ingredient(s). When administered intravenously, the most preferred doses will range from about 0.001 to about 10 μg/kg/hour during constant rate infusion. Advantageously, treatment may comprise administration of such compounds and compositions in a single daily dose, or the total daily dosage may be administered in divided doses of two, three or four times daily (e.g. twice daily with reference to the doses described herein, such as a dose of 10 mg, 20 mg, 30 mg or 40 mg twice daily, or 10 μg, 20 μg, 30 μg or 40 μg twice daily).

In any event, the skilled person (e.g. the physician) will be able to determine the actual dosage which will be most suitable for an individual patient, which is likely to vary with the route of administration, the type and severity of the condition that is to be treated, as well as the species, age, weight, sex, renal function, hepatic function and response of the particular patient to be treated. The above-mentioned dosages are exemplary of the average case; there can, of course, be individual instances where higher or lower dosage ranges are merited, and such are within the scope of this invention.

As described herein above, the skilled person will understand that treatment with compounds of the first aspect of the invention may further comprise (i.e. be combined with) further (i.e. additional/other) treatment(s) for the same condition. In particular, treatment with compounds of the invention may be combined with other means for the treatment of hyperglycaemia or a disoder characterized by hyperglycaemia(as defined herein, such as type 2 diabetes), such as treatment with one or more other therapeutic agent that is useful in the treatment of hyperglycaemia or a disoder characterized by hyperglycaemia(as defined herein, such as type 2 diabetes).

In particular embodiments of the fourth and fifth aspects of the invention, the pharmaceutical composition may further comprise one or more additional (i.e. other) therapeutic agent.

In more particular embodiments, the one or more additional therapeutic agent is an agent for the treatment of type 2 diabetes as known to those skilled in the art, such as metformin, sulfonylureas (e.g. carbutamide, acetohexamide, chlorpropamide, tolbutamide. glipizide (glucotrol), gliclazide, glibenclamide, glyburide (Micronase), glibornuride, gliquidone, glisoxepide, glyclopyramide, glimepiride (Amaryl), glimiprime, JB253 or JB558), thiazolidinediones (e.g. pioglitazone, rosiglitazone (Avandia), lobeglitazone (Duvie) and troglitazone (Rezulin)), dipeptidyl peptidase-4 inhibitors (e.g. sitagliptin, vildagliptin, saxagliptin, linagliptin, anagliptin, teneligliptin, alogliptin, trelagliptin, gemigliptin, dutogliptin and omarigliptin), SGLT2 inhibitors (e.g. dapagliflozin, empagliflozin, canagliflozin, ipragliflozin, tofogliflozin, sergliflozin etabonate, remogliflozin etabonate, and ertugliflozin), and glucagon-like peptide-1 (GLP-1) analogues.

The skilled person will understand that combinations of therapeutic agents may also described as a combination product and/or provided as a kit-of-parts.

In a sixth aspect of the invention, there is provided a combination product comprising:
(A) a compound as defined in the first aspect of the invention; and
(B) one or more additional therapeutic agent,
   wherein each of components (A) and (B) is formulated in admixture, optionally with one or more a pharmaceutically-acceptable adjuvant, diluent or carrier.

In a seventh aspect of the invention, there is provided a kit-of-parts comprising:
(a) a compound as defined in the first (or second and/or third) aspect of the invention, (or a pharmaceutical composition comprising the same) or a pharmaceutical composition as defined in the fourth or fifth aspect of the invention; and
(b) one or more other therapeutic agent, optionally in admixture with one or more pharmaceutically-acceptable adjuvant, diluent or carrier,
   which components (a) and (b) are each provided in a form that is suitable for administration in conjunction with the other.

In particular embodiments (e.g. of the sixth and seventh aspects of the invention), the additional therapeutic agent is a therapeutic agent that is useful for the treatment of hyperglycaemia or a disorder characterized by hyperglycaemia (e.g. type 2 diabetes), as known to those skilled in the art (such as those described herein).

For example, in particular embodiments of the fourth to fifth aspects of the invention, the additional therapeutic agent is an agent that:
(i) is capable of reducing blood sugar levels; and/or
(ii) is an insulin sensitizer; and/or
(iii) is able to enhance insulin release,
   which agents will be readily identified by those skilled in the art and include, in particular, such therapeutic agents that are commercially available (e.g. agents that the subject of a marketing authorization in one or more territory, such as a European or US marketing authorization).

The skilled person will understand that references to therapeutic agents capable of reducing blood glucose levels may refer to compounds capable of reducing levels of blood by at least 10% (such as at least 20%, at least 30% or at least 40%, for example at least 50%, at least 60%, at least 70% or at least 80%, e.g. at least 90%) when compared to the blood glucose levels prior to treatment with the relevant compound.

In alternative embodiments of the sixth and seventh aspects of the invention, the additional therapeutic agent is an agent for the treatment or prevention of a non-alcoholic fatty liver disease (such as NASH), which agents will be readily identified by those skilled in the art and include, in particular, such therapeutic agents that are commercially available (e.g. agents that the subject of a marketing authorization in one or more territory, such as a European or US marketing authorization).

In alternative embodiments of the sixth and seventh aspects of the invention, the additional therapeutic agent is an agent for treating a disease or disorder the treatment of which is mediated by activation of the _β₂ adrenergic receptor, which diseases and disorders will include those described herein, and which agents will be readily identified by those skilled in the art and include, in particular, such therapeutic agents that are commercially available (e.g. agents that the subject of a marketing authorization in one or more territory, such as a European or US marketing authorization).

Preparation of Compounds/Compositions

Pharmaceutical compositions/formulations, combination products and kits as described herein may be prepared in accordance with standard and/or accepted pharmaceutical practice.

Thus, in a further aspect of the invention there is provided a process for the preparation of a pharmaceutical composition/formulation, as hereinbefore defined, which process comprises bringing into association a compound of the invention, as hereinbefore defined, with one or more pharmaceutically-acceptable adjuvant, diluent or carrier.

In further aspects of the invention, there is provided a process for the preparation of a combination product or kit-of-parts as hereinbefore defined, which process comprises bringing into association a compound of the invention, as hereinbefore defined, or a pharmaceutically acceptable salt thereof with the other therapeutic agent that is useful in the treatment of hyperglycaemia or a disorder characterized by hyperglycaemia (e.g. type 2 diabetes), and at least one pharmaceutically-acceptable adjuvant, diluent or carrier.

As used herein, references to bringing into association will mean that the two components are rendered suitable for administration in conjunction with each other.

Thus, in relation to the process for the preparation of a kit of parts as hereinbefore defined, by bringing the two components “into association with” each other, we include that the two components of the kit of parts may be:
(i) provided as separate formulations (i.e. independently of one another), which are subsequently brought together for use in conjunction with each other in combination therapy; or
(ii) packaged and presented together as separate components of a “combination pack” for use in conjunction with each other in combination therapy.

Compounds as defined in the first (and, therefore, second and third) aspect of the invention (i.e. compounds of the invention) may be prepared in accordance with techniques that are well known to those skilled in the art, such as those described in the examples provided hereinafter.

For example, there is provided a process for the preparation of a compound of formula I, or a pharmaceutically acceptable salt thereof, as defined in the first aspect of the invention (which may be utilised in the preparation of, for example, a compound as defined in the second aspect of the invention), which process comprises:
(i) reaction of a compound of formula II

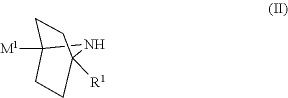

wherein R¹ is as defined herein, and wherein M¹ represents a suitable metal or metal halide, with a compound of formula III

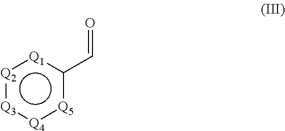

wherein Q¹ to Q⁵ are as defined herein, under conditions known to those skilled in the art;
(ii) reaction of a compound of formula IV

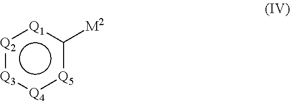

wherein Q¹ to Q⁵ are as defined herein, and wherein M2 represents a suitable metal or metal halide, with a compound of formula V

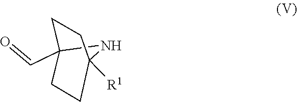

wherein RX¹ is as defined herein, under conditions known to those skilled in the art;
(iii) for compounds wherein at least one X¹ or X² is present and represents —OH, deprotection of a compound of formula VI

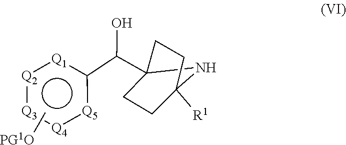

wherein Q¹ to Q⁵, X¹, X² and R¹ are as defined hereinabove, PG¹ represents a suitable protecting group as known to those skilled in the art (e.g. benzyl, alkyl groups, silyl protecting groups, e.g. TMS or TBDMS, acyl groups, e.g. acetyl or benzoyl, or sulphonyl groups, e.g. trifluoromethylsulphonyl or tosyl) under conditions known to those skilled in the art (for example, in the case of a benzyl protecting group, in the presence of hydrogen and a suitable catalyst or a suitable acid; in the case of alkyl, such as methyl, in the presence of BBr₃, HBr or alkyl sulphides; in the case of silyl, in the presence of CsF, Bu₄NF and the like; in the case of acyl, by basic hydrolysis (NaOH, K₂CO₃, etc));
(iv) for compounds wherein at least one X¹ or X² is present and represents —NH₂, deprotection of a compound of formula VII

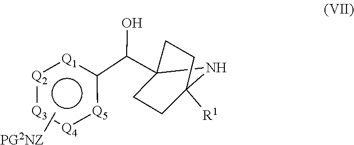

wherein Q¹ to Q⁵, X¹, X² and R¹ are as defined hereinabove, and Z represents H or PG³, wherein PG² and PG³ each represents a suitable protecting group as known to those skilled in the art (e.g. a carbamate protecting group, such as tert-butyloxycarbonyl (Boc), fluorenylmethyloxycarbonyl (Fmoc) or carboxybenzyl (Cbz), an amide protecting group, such as acetyl and benzoyl, or sulphonyl groups, e.g. trifluoromethylsulphonyl or tosyl), under conditions known to those skilled in the art (for example in the case of Boc, in the presence of a suitable acid (e.g. in the case of a carbamate, trifluoroacetic acid or HCl; in the case of sulphonyl, basic hydrolysis (e.g. in the presence of NaOH or K₂CO₃));
(v) for compounds wherein at least one X¹ or X² is present and represents NH₂, reduction of a compound of formula VIII

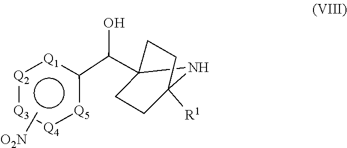

wherein Q¹ to Q⁵, X¹, X² and R¹ are as defined hereinabove, under conditions known to those skilled in the art (for example, by hydrogenation, such as hydrogenation using hydrogen gas and a suitable catalyst as known to those skilled in the art,(e.g. Pd—C, PtO₂, Raney-Nickel), Fe or Zn in acidic media (e.g. AcOH), borohydrides together with a suitable catalyst (e.g. NaBH₄ and Raney-Nickel), or agents such as SnCl₂, TiCl₃, SmI₂, and the like). Those skilled in the art will understand that certain functional groups, such as the essential —OH and/or the —NHR¹ groups) may need to be protected (and to deprotected) one or more times during the reaction, which protections (and deprotections) may be performed using techniques known to those skilled in the art;
(vi) deprotection of a compound of formula IX

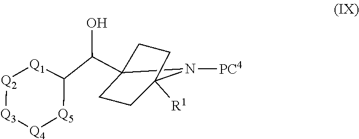

wherein Q¹ to Q⁵ and R¹ are as defined hereinabove, and PG⁴ represents a suitable protecting group as known to those skilled in the art (e.g. a carbamate protecting group, such as tert-butyloxycarbonyl (Boc), fluorenylmethyloxycarbonyl (Fmoc) or carboxybenzyl (Cbz), an amide protecting group, such as acetyl and benzoyl, or sulphonyl groups, e.g. trifluoromethylsulphonyl or tosyl), under conditions known to those skilled in the art (for example in the case of Boc, in the presence of a suitable acid (e.g. in the case of a carbamate, trifluoroacetic acid or HCl; in the case of sulphonyl, basic hydrolysis (e.g. in the presence of NaOH or K₂CO₃));
(vii) for compounds wherein at least one X¹ or X² is present and represents NH₂, reduction of a compound of formula X

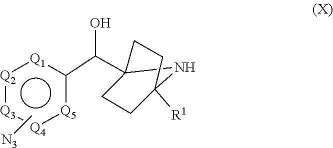

wherein Q¹ to Q⁵, X¹, X² and R¹ are as defined hereinabove, under conditions known to those skilled in the art (for example, by reduction of the —N₃ group, such as by reaction with a suitable reducing agent, e.g. SmI₂, and the like);
(viii) reduction of a compound of formula XI

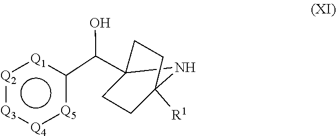

wherein Q¹ to Q⁵ and R¹ are as defined hereinabove and Y¹ represents H or PG⁵, wherein PG⁵ is a suitable protecting group as known to those skilled in the art (e.g. —C(O)OtBu or —SO₂CH₃), either:
with a suitable reduction agent as known to those skilled in the art (such as NaBH₄ or LiAlH₄) or by hydrogenation in the presence of a suitable catalyst; or
where the carbon bound to the essential OH group in compounds of the invention (i.e. carbon (a) as defined herein) is chiral, in the presence of a suitable catalyst (such as a complex between (1S, 2S)-(+)-N-(4-toluenesulphonyl)-1,2-diphenylethylene diamine and [Ru(cymene)Cl₂]₂)) and in the presence of hydrogen or a suitable hydrogen donor (such as formic acid) and optionally in the presence of a base (e.g. Et₃N) and in the presence of a suitable solvent (such as CH₂Cl₂).

Compounds of formulae II, III, IV, V, VI, VII, VIII and IX, X and XI are either commercially available, are known in the literature, or may be obtained either by analogy with the processes described herein, or by conventional synthetic procedures, in accordance with standard techniques, from available starting materials (e.g. appropriately substituted benzaldehydes, styrenes or phenacyl bromides (or phenacylchloride, and the like) using appropriate reagents and reaction conditions. In this respect, the skilled person may refer to inter alia “Comprehensive Organic Synthesis” by B. M. Trost and I. Fleming, Pergamon Press, 1991. Further references that may be employed include “Science of Synthesis”, Volumes 9-17 (Hetarenes and Related Ring Systems), Georg Thieme Verlag, 2006.

The substituents X¹, X² and R¹, as hereinbefore defined, may be modified one or more times, after or during the processes described above for preparation of compounds of formula I by way of methods that are well known to those skilled in the art. Examples of such methods include substitutions, reductions, oxidations, dehydrogenations, alkylations, dealkylations, acylations, hydrolyses, esterifications, etherifications, halogenations and nitrations. The precursor groups can be changed to a different such group, or to the groups defined in formula I, at any time during the reaction sequence. The skilled person may also refer to “Comprehensive Organic Functional Group Transformations” by A. R. Katritzky, O. Meth-Cohn and C. W. Rees, Pergamon Press, 1995 and/or “Comprehensive Organic Transformations” by R. C. Larock, Wiley-VCH, 1999.

Such compounds may be isolated from their reaction mixtures and, if necessary, purified using conventional techniques as known to those skilled in the art. Thus, processes for preparation of compounds of the invention as described herein may include, as a final step, isolation and optionally purification of the compound of the invention (e.g. isolation and optionally purification of the compound of formula I).

The skilled person will understand that compounds of formula I having specific stereochemistry may be provided by reacting suitable starting materials having the required stereochemistry in processes as described herein. Further, the skilled person will understand that suitable starting materials having the required stereochemistry may be prepared by analogy with the processes described herein.

It will be appreciated by those skilled in the art that, in the processes described above and hereinafter, the functional groups of intermediate compounds may need to be protected by protecting groups. The protection and deprotection of functional groups may take place before or after a reaction in the above-mentioned schemes.

Protecting groups may be applied and removed in accordance with techniques that are well known to those skilled in the art and as described hereinafter. For example, protected compounds/intermediates described herein may be converted chemically to unprotected compounds using standard deprotection techniques. The type of chemistry involved will dictate the need, and type, of protecting groups as well as the sequence for accomplishing the synthesis. The use of protecting groups is fully described in “Protective Groups in Organic Synthesis”, 3rd edition, T. W. Greene & P. G. M. Wutz, Wiley-Interscience (1999).

Compounds as described herein (in particular, compounds as defined in the first and, therefore, second and third aspects of the invention) may have the advantage that they may be more efficacious than, be less toxic than, be longer acting than, be more potent than, produce fewer side effects than, be more easily absorbed than, and/or have a better pharmacokinetic profile (e.g. higher oral bioavailability and/or lower clearance) than, and/or have other useful pharmacological, physical, or chemical properties over, compounds known in the prior art, whether for use in the above-stated indications or otherwise. In particular, such compounds may have the advantage that they are more efficacious and/or exhibit advantageous properties in vivo.

Without wishing to be bound by theory, compounds as described herein are thought to be potent agonists of the β₂-adrenergic receptor, which allows for increased glucose uptake in skeletal muscle cells.

In addition, compounds as described herein are thought to be agonists of the β₂-adrenergic receptor without (or with only a relatively minimal effect in, such as a relatively lesser effect in (when compared to the effect in inducing increased glucose uptake)) inducing cAMP production. It is thought that this allows for the increased glucose uptake in skeletal muscle cells with lower levels of side effects than would result from other treatments. Further, combining compounds as described herein with therapeutic agents that are able to decrease blood glucose levels is thought to provide an effective combination therapy.

### EXAMPLES

The present invention is illustrated by way of the following examples.

Chemicals and reagents were obtained from commercial suppliers and were used as received unless otherwise stated. All reactions involving moisture sensitive reagents were performed in oven or flame dried glassware under a positive pressure of nitrogen or argon.

### ABBREVIATIONS

Abbreviations as used herein will be known to those skilled in the art. In particular, the following abbreviations may be used herein.
aq aqueous
BuLi butyl lithium
DMSO dimethylsulfoxide
EtOH ethanol
EtOAc ethyl acetate
iPrOH isopropanol
rt room temperature
sat saturated
THF tetrahydrofuran
TM EDA N,N,N′,N′-tetramethylethylenediamine

### EXAMPLE COMPOUNDS

In the event that there is a discrepancy between nomenclature and the structure of compounds as depicted graphically, it is the latter that presides (unless contradicted by any experimental details that may be given and/or unless it is clear from the context).

### Example 1: (R)-(3-Fluorophenyl)(4-methyl-7-azabicyclo[2.2.1]heptan-1-yl)methanol hydrochloride

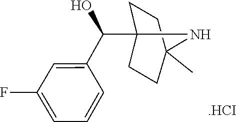

(a) tert-Butyl 7-azabicyclo[2.2.1]heptane-7-carboxylate

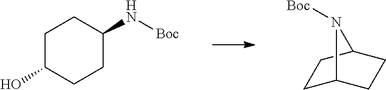 [SMILES: CC(C)(C)OC(=O)N[C@H]1CC[C@H](O)CC1 ; CC(C)(C)OC(=O)N1C2CCC1CC2]

Methanesulfonyl chloride (3.20 mL, 41.8 mmol) was added to a stirred ice-cooled mixture of (trans) tert-butyl-4-hydroxycyclohexyl)carbamate (6 g, 27.9 mmol), Et₃N (5.80 mL, 41.9 mmol) and CH₂C₁₂ (200 mL). The cooling bath was removed and the mixture was stirred at rt for X¹ h. Et₃N (5.80 mL, 41.9 mmol) and methanesulfonyl chloride (3.20 mL, 41.8 mmol) were added at rt and stirring was continued for X¹ h. Additional portions of Et₃N (5.80 mL, 41.9 mmol) and methanesulfonyl chloride (3.20 mL, 41.8 mmol) were added at rt and stirring was continued for 30 min. NaHCO₃ (aq, sat) was carefully added and the mixture was extracted with CH₂Cl₂. The organic phase was washed with water and brine, dried (Mg₂SO₄) and concentrated. The residue was dissolved in THF (200 mL), cooled in an ice bath and tBuOK (9.38 g, 83.6 mmol) was added in portions. The cooling bath was removed and the mixture was stirred at rt overnight. Crushed ice was added and the mixture was stirred until the ice melted. The mixture was extracted with Et₂O and the layers were separated. The aq phase was extracted with Et₂O and the combined organic phases were washed with water and brine, dried (Na₂SO₄) and concentrated. The residue was purified by chromatography to give the sub-title compound (4.16 g, 76%).

(b) tert-Butyl 1-methyl-7-azabicyclo[2.2.1]heptane-7-carboxylate

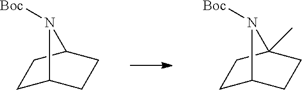

sec-BuLi (1.3 M in cyclohexane, 8.8 mL, 11.4 mmol) was added dropwise to a stirred ice-cooled solution of tert-butyl 7-azabicyclo[2.2.1]heptane-7-carboxylate (1.5 g, 7.60 mmol) and TMEDA (1.7 mL, 11.4 mmol) in Et₂O (15 mL). The cooling bath was removed and the mixture was stirred at rt for 5 min. The mixture was again cooled in an ice-bath and MeI (0.95 mL, 15.2 mmol) was added dropwise. After stirring for 2 h at rt NH₄Cl (aq, sat) was added. The aq phase was extracted with Et₂O and the combined organic phases were washed with water and brine, dried (Na₂SO₄) and concentrated. The residue was purified by chromatography to give the sub-title compound (1.45 g, 90%).

(c) tert-Butyl 1-((R)-(3-fluorophenyl)(hydroxy)methyl)-4-methyl-7-azabicyclo -[2.2.1]heptane-7-carboxylate

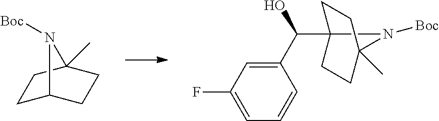 [SMILES: CC(C)(C)OC(=O)N1C2CCC1(C)CC2 ; CC(C)(C)OC(=O)N1C2(C)CCC1([C@H](O)c1cccc(F)c1)CC2]

sec-BuLi (1.3 M in cyclohexane, 0.67 mL, 0.87 mmol) was added dropwise to a stirred ice-cooled solution of tert-butyl 1-methyl-7-azabicyclo[2.2.1]heptane-7-carboxylate (122 mg, 0.58 mmol) and TMEDA (130 μL, 0.87 mmol) in Et₂O (1.5 mL). The cooling bath was removed and the mixture was stirred at rt for 5 min. The mixture was again cooled in an ice-bath and 3-fluorobenzaldehyde (0.12 mL, 1.16 mmol) was added dropwise. After stirring for 1 h at rt NH₄Cl (aq, sat) was added. The aq phase was extracted with Et₂O and the combined organic phases were washed with brine, dried (Na₂SO₄) and concentrated. The residue was purified by chromatography on silica gel followed by chiral chromatography (column: DAICEL CHIRALPAK ID-5 μm, 250×30 mm, system: 2% iPrOH and 5% CH₂Cl₂ in heptane, 40 mL/min, λ 260 nm) to give the sub-title compound (55 mg, 28%) along with its (S)-enantiomer (60 mg, 31%).

(d) (1R)-(3-Fluorophenyl)(4-methyl-7-azabicyclo[2.2.1]heptan-1-yl)methanol hydrochloride

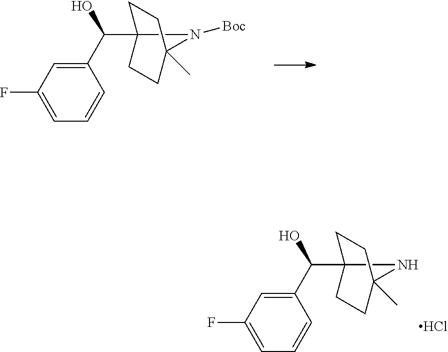 [SMILES: CC(C)(C)OC(=O)N1C2(C)CCC1([C@H](O)c1cccc(F)c1)CC2 ; CC12CCC([C@H](O)c3cccc(F)c3)(CC1)N2 ; Cl]

A solution of NaOH (45 mg, 1.13 mmol) in H₂O (1.5 mL) was added to a solution of tert-butyl 1-(R)-(3-fluorophenyl)(hydroxy)methyl)-4-methyl-7-azabicyclo[2.2.1]-heptane -7-carboxylate (46 mg, 0.14 mmol) in EtOH (1.5 mL). The mixture was stirred at 120° C. for 16 h and concentrated. The residue was extracted with EtOAc and the combined extracts washed with water and brine, dried (Na₂SO₄) and concentrated. The residue was dissolved in Et₂O (3 mL) and HCl in Et₂O (2 M, 68 μL, 0.14 mmol) was added dropwise. The mixture was held at −20° C. for 45 min and the solid was collected to give the title compound (23 mg, 75%).

¹H NMR (400 MHz, D₂O): δ 7.51-7.38 (m, 1H), 7.29-7.09 (m, 3H), 5.10 (s, 1H), 2.38-2.21 (m, 1H), 2.20-2.03 (m, 1H), 2.02-1.81 (m, 4H), 1.73-1.59 (m, 2H), 1.54 (s, 3H).

### Example 2: (S)-(3-Fluorophenyl)(4-methyl-7-azabicyclo[2.2.1]heptan-1-yl)methanol hydrochloride

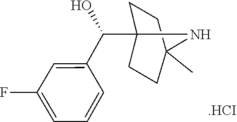

The title compound was prepared from tert-butyl 1-((S)-(3-fluorophenyl) -(hydroxy)methyl)-4-methyl-7-azabicyclo[2.2.1]heptane-7-carboxylate, see Example 1, Step (c) in accordance with the procedure in Example 1, Step (d).

The ¹H NMR spectrum was identical to the one for the (R) enantiomer in Example 1.

### Example 3: (R)-(2-Fluorophenyl)(4-methyl-7-azabicyclo[2.2.1]heptan-1-yl)methanol hydrochloride

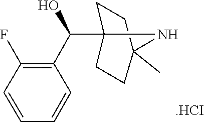

The title compound was prepared in accordance with the procedures in Example 1, using 2-fluorobenzaldehyde in step (c).

¹H NMR (400 MHz, D₂O): δ 7.59-7.50 (m, 1H), 7.49-7.39 (m, 1H), 7.35-7.26 (m, 1H), 7.21 (ddd, J=10.9, 8.3, 1.2 Hz, 1H), 5.44 (s, 1H), 2.34-2.20 (m, 1H), 2.20-2.07 (m, 1H), 2.02-1.85.

### Example 4: (S)-(2-Fluorophenyl)(4-methyl-7-azabicyclo[2.2.1]heptan-1-yl)methanol hydrochloride

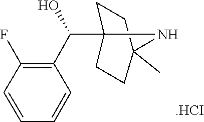

The title compound was prepared in accordance with the procedures in Example 1, using 2-fluorobenzaldehyde in step (c).

The ¹H NMR spectrum was identical to the one for the (R) enantiomer in Example 3.

### Example 5: (R)-(3-Chlorophenyl)(4-methyl-7-azabicyclo[2.2.1]heptan-1-yl)methanol hydrochloride

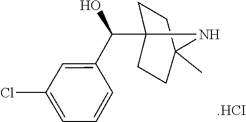

The title compound was prepared in accordance with the procedures in Example 1, using 3-chlorobenzaldehyde in step (c).

¹H NMR (400 MHz, D₂O): δ 7.51-7.39 (m, 3H), 7.38-7.31 (m, 1H), 5.08 (s, 1H), 2.36-2.21 (m, 1H), 2.18-2.05 (m, 1H), 2.02-1.84 (m, 4H), 1.73-1.59 (m, 2H), 1.54 (s, 3H).

### Example 6: (S)-(3-Chlorophenyl)(4-methyl-7-azabicyclo[2.2.1]heptan-1-yl)methanol hydrochloride

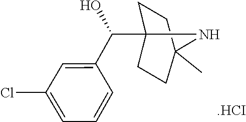

The title compound was prepared in accordance with the procedures in Example 1, using 3-chlorobenzaldehyde in step (c).

The ¹H NMR spectrum was identical to the one for the (R) enantiomer in Example 5.

### Example 7: (R)-(2-Chlorophenyl)(4-methyl-7-azabicyclo[2.2.1]heptan-1-yl)methanol hydrochloride

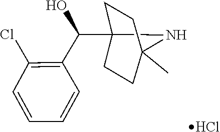 [SMILES: CC12CCC([C@H](O)c3ccccc3Cl)(CC1)N2 ; Cl]

The title compound was prepared in accordance with the procedures in Example 1, using 2-chlorobenzaldehyde in step (c).

¹H NMR (400 MHz, D₂O): δ 7.64-7.57 (m, 1H), 7.53-7.48 (m, 1H), 7.48-7.35 (m, 2H), 5.64 (s, 1H), 2.37-2.20 (m, 2H), 2.03-1.82 (m, 5H), 1.77-1.64 (m, 1H), 1.69 (ddd, J=12.7, 9.7, 4.7 Hz, 1H), 1.54 (s, 3H).

### Example 8: (5)-(2-Chlorophenyl)(4-methyl-7-azabicyclo[2.2.1]heptan-1-yl)methanol hydrochloride

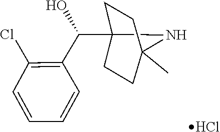 [SMILES: CC12CCC([C@@H](O)c3ccccc3Cl)(CC1)N2 ; Cl]

The title compound was prepared in accordance with the procedures in Example 1, using 2-chlorobenzaldehyde in step (c).

The ¹H NMR spectrum was identical to the one for the (R) enantiomer in Example 7.

### Example 9: (R)-(3-fluoropyridin-4-yl)(4-methyl-7-azabicydo[2.2.1]heptan-1-yl)-methanol

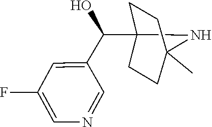 [SMILES: CC12CCC([C@H](O)c3cncc(F)c3)(CC1)N2]

(a) tert-Butyl 1-((R)-(5-fluoropyridin-3-yl)(hydroxy)methyl)-4-methyl-7-azabicyclo[2.2.1]heptane-7-carboxylate

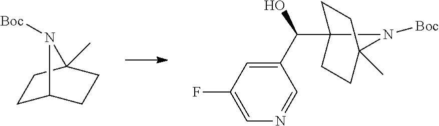 [SMILES: CC(C)(C)OC(=O)N1C2CCC1(C)CC2 ; CC(C)(C)OC(=O)N1C2(C)CCC1([C@H](O)c1cncc(F)c1)CC2]

sec-BuLi (1.3 M in cyclohexane, 0.83 mL, 1.08 mmol) was added dropwise to a stirred ice-cooled solution of tert-butyl 1-methyl-7-azabicyclo[2.2.1]heptane-7-carboxylate (190 mg, 0.90 mmol) and TMEDA (0.16 mL, 1.08 mmol) in Et₂O (2.3 mL). The cooling bath was removed and the mixture was stirred at rt for 5 min and cooled to −78° C. 3-Fluoro-5-formylpyridine (0.20 mL, 1.80 mmol) was added dropwise. After stirring for 1 h at rt NH₄Cl (aq, sat) was added. The aq phase was extracted with Et₂O and the combined organic phases were washed with brine, dried (Na₂SO₄) and concentrated. The residue was purified by chromatography on silica gel followed by chiral chromatography (column: DAICEL CHIRALPAK IC-5 μm, 250×30 mm, system: 5% THF in heptane, 40 mL/min, λ 260 nm) to give the sub-title compound (21 mg, 7%) along with its (S)-enantiomer (52 mg, 17%).

(b) (R)-(3-Fluoropyridin-4-yl)(4-methyl-7-azabicyclo[2.2.1]heptan-1-yl)methanol

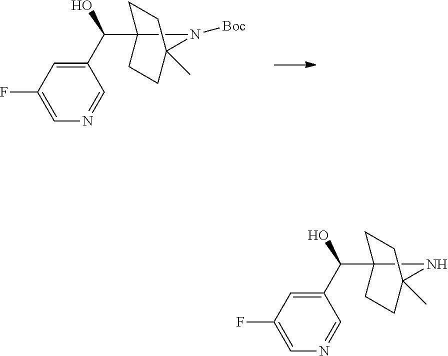 [SMILES: CC(C)(C)OC(=O)N1C2(C)CCC1([C@H](O)c1cncc(F)c1)CC2 ; CC12CCC([C@H](O)c3cncc(F)c3)(CC1)N2]

A solution of NaOH (55 mg, 1.37 mmol) in H₂O (2 mL) was added to a solution of tert-butyl 1-((R)-(5-fluoropyridin-3-yl)(hydroxy)methyl)-4-methyl-7-azabicyclo[2.2.1]-heptane -7-carboxylate (46 mg, 0.14 mmol) in EtOH (2 mL). The mixture was stirred at 120° C. for 16 h and concentrated. The residue was extracted with EtOAc and the combined extracts washed with water and brine, dried (Na₂SO₄) and concentrated to give the title compound (25 mg, 77%).

¹H NMR (400 MHz, CDCl₃) δ 8.43-8.32 (m, 2H), 7.58-7.45 (m, 1H), 5.05 (s, 1H), 3.30 (br s, 2H), 2.04-1.88 (m, 1H), 1.79-1.45 (m, 5H), 1.42-1.29 (m, 4H), 1.14 (ddd, J=11.8, 8.9, 5.0 Hz, 1H).

### Example 10: (S)-(3-fluoropyridin-4-yl)(4-methyl-7-azabicydo[2.2.1]heptan-1-yl)-methanol

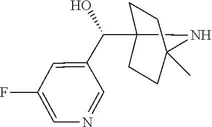 [SMILES: CC12CCC([C@@H](O)c3cncc(F)c3)(CC1)N2]

The title compound was prepared from tert-butyl 1-((S)-(5-fluoropyridin-3-yl) -(hydroxy)methyl)-4-methyl-7-azabicyclo[2.2.1]heptane-7-carboxylate, see Example 9, Step (a) in accordance with the procedure in Example 9, Step (b).

The ¹H NMR spectrum was identical to the one for the (R) enantiomer in Example 9.

Example 11: (R)-(3-Fluoropyridin-4-yl)(4-methyl-7-azabicyclo[2.2.1]heptan-1-yl)-methanol

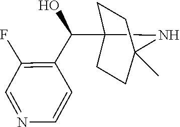 [SMILES: CC12CCC([C@H](O)c3ccncc3F)(CC1)N2]

(a) tert-Butyl 1-((R)-(3-fluoropyridin-4-yl)(hydroxy)methyl)-4-methyl-7-azabicyclo[2.2.1]heptane-7-carboxylate

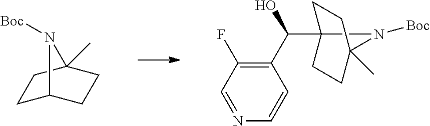 [SMILES: CC(C)(C)OC(=O)N1C2CCC1(C)CC2 ; CC(C)(C)OC(=O)N1C2(C)CCC1([C@H](O)c1ccncc1F)CC2]

sec-BuLi (1.3 M in cyclohexane, 1.20 mL, 1.41 mmol) was added dropwise to a stirred ice-cooled solution of tert-butyl 1-methyl-7-azabicyclo[2.2.1]heptane-7-carboxylate (198 mg, 0.94 mmol) and TMEDA (0.21 mL, 1.41 mmol) in Et₂O (2.3 mL). The cooling bath was removed and the mixture was stirred at rt for 5 min and cooled to −78° C. 3-Fluoroisonicotinaldehyde (0.15 mL, 1.63 mmol) was added dropwise. After stirring for 1 h at rt NH₄Cl (aq, sat) was added. The aq phase was extracted with Et₂O and the combined organic phases were washed with brine, dried (Na₂SO₄) and concentrated. The residue was purified by chromatography on silica gel followed by chiral chromatography (column: DAICEL CHIRALPAK IG-5 μm, 250×30 mm, system: 25% iPrOH in heptane, 40 mL/min, λ 260 nm) to give the sub-title compound (86 mg, 27%) along with its (S)-enantiomer (85 mg, 27%).

(b) (R)-(3-Fluoropyridin-4-yl)(4-methyl-7-azabicyclo[2.2.1]heptan-1-yl)methanol

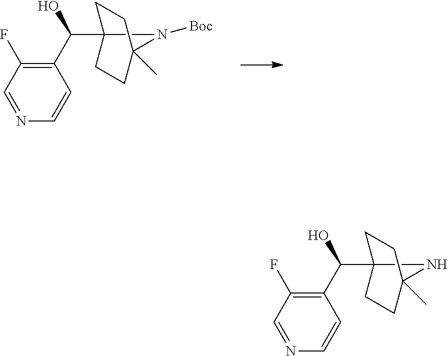 [SMILES: CC(C)(C)OC(=O)N1C2(C)CCC1([C@H](O)c1ccncc1F)CC2 ; CC12CCC([C@H](O)c3ccncc3F)(CC1)N2]

The title compound was prepared from tert-butyl 1-((R)-(3-fluoropyridin-4-yl) -(hydroxy)methyl)-4-methyl-7-azabicyclo[2.2.1]heptane-7-carboxylate in accordance with the procedure in Example 9, Step (b).

¹H NMR (400 MHz, CDCl₃) δ 8.52-8.32(m, 2H), 7.60-7.46 (m, 1H), 5.47-7.32 (s, 1H), 3.37-2.25 (br s, 2H), 2.07-1.83 (m, 2H), 1.75-1.48 (m,4H), 1.47-1.30 (m, 4H), 1.28-1.12 (m, 1H).

### Example 12: (S)-(3-Fluoropyridin-4-yl)(4-methyl-7-azabicydo[2.2.1]heptan-1-yl)-methanol

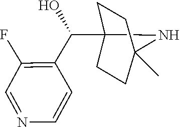 [SMILES: CC12CCC([C@@H](O)c3ccncc3F)(CC1)N2]

The title compound was prepared from tert-butyl 1-((S)-(3-fluoropyridin-4-yl)(hydroxy)methyl)-4-methyl-7-azabicyclo[2.2.1]heptane-7-carboxylate (see Example 11, step (a)) in accordance with the procedure in Example 9, Step (b).

The 1H NMR spectrum was identical to the one for the (R) enantiomer in Example 11.

### Example 13: (R)-(3-Fluorophenyl)(4-propyl-7-azabicyclo[2.2.1]heptan-1-yl)methanol hydrochloride

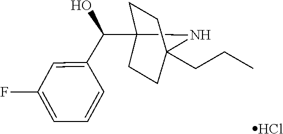 [SMILES: CCCC12CCC([C@H](O)c3cccc(F)c3)(CC1)N2 ; Cl]

The title compound was prepared in accordance with the procedures in Example 1, using n-propyl iodide in Step (b).

¹H NMR (400 MHz, D₂O) δ 7.49-7.40 (m, 1H), 7.28-7.10 (m, 3H), 5.10 (s, 1H), 2.37-2.23 (m, 1H), 2.16-2.02 (m, 1H), 2.02-1.78 (m, 6H), 1.71-1.56 (m, 2H), 1.48-1.32 (m, 2H), 0.96 (t, J=7.3 Hz, 1H).

### Example 14: (S)-(3-Fluorophenyl)(4-propyl-7-azabicyclo[2.2.1]heptan-1-yl)methanol hydrochloride

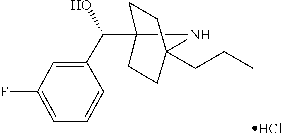 [SMILES: CCCC12CCC([C@@H](O)c3cccc(F)c3)(CC1)N2 ; Cl]

The title compound was prepared in accordance with the procedures in Example 1, using n-propyl iodide in Step (b).

The ¹H NMR spectrum was identical to the one for the (R) enantiomer in Example 13.

### Example 15: (S)-(3-Fluorophenyl)(4-(methoxymethyl)-7-azabicyclo[2.2.1]heptan -1-yl)methanol hydrochloride

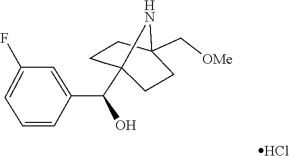 [SMILES: COCC12CCCCC([C@@H](O)c3cccc(F)c3)(CC1)N2 ; Cl]

(a) 7-(tert-Butoxycarbonyl)-7-azabicyclo[2.2.1]heptane-1-carboxylic acid

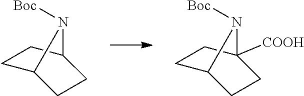 [SMILES: CC(C)(C)OC(=O)N1C2CCC1CC2 ; CC(C)(C)OC(=O)N1C2CCC1(C(=O)O)CC2]

sec-BuLi (1.23 M in cyclohexane/hexane (92/8), 4.4 ml, 5.45 mmol) was added to a stirred ice-cooled solution of tert-butyl-7-azabicyclo[2.2.1]heptane-7-carboxylate, see Example 1, Step (a), (0.82 g, 4.14 mmol) and TMEDA (0.93 ml, 6.20 mmol) in Et₂O (8.2 mL). The cooling bath was removed and the mixture was stirred at rt for 5 min and again cooled in the ice-bath. CO₂was bubbled through the mixture for 7 min and the mixture was stirred for 30 min at 0° C., allowed to warm to rt and washed with H₂O. The layers were separated and the aq layer acidified with HCl (aq, 2 M) until pH ˜3 and extracted with EtOAc. The combined extracts were washed with brine, dried (MgSO₄) and concentrated to give the sub-title compound (0.77 g, 77%).

(b) 7-(tert-Butyl) 1-methyl-7-azabicyclo[2.2.1]heptane-1,7-dicarboxylate

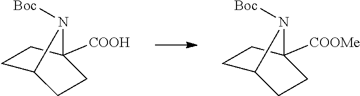 [SMILES: CC(C)(C)OC(=O)N1C2CCC1(C(=O)O)CC2 ; COC(=O)C12CCC(CC1)N2C(=O)OC(C)(C)C]

K₂CO₃ (1.15 g, 8.29 mmol) was added in portions to a solution of 7-(tert -butoxycarbonyl)-7-azabicyclo[2.2.1]heptane-1-carboxylic acid (0.50 g, 2.07 mmol) in DMF (6.2 ml) at rt. The mixture was stirred at rt for 15 min whereafter MeI (0.15 mL, 2.89 mmol) was added. The mixture was stirred at rt for 16 h, diluted with H₂O and extracted with EtOAc. The combined extracts were washed with H₂O, NaHCO₃ (aq, sat) and brine, dried (MgSO₄) and concentrated. The residue was purified by chromatography to give the sub-title compound (0.42 g, 79%).

(c) tert-Butyl 1-(hydroxymethyl)-7-azabicyclo[2.2.1]heptane-7-carboxylate

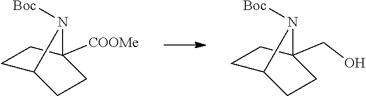 [SMILES: COC(=O)C12CCC(CC1)N2C(=O)OC(C)(C)C ; CC(C)(C)OC(=O)N1C2CCC1(CO)CC2]

Lithium triethylborohydride (1 M in THF, 3.5 mL, 3.5 mmol) was added dropwise to a stirred ice-cooled solution of 7-(tert-butyl) 1-methyl-7-azabicyclo[2.2.1]heptane -1,7-dicarboxylate (0.41 g, 1.61 mmol) in THF (11 mL). The mixture was stirred at 0° C. for 1 h and another portion of lithium triethylborohydride (1.77 mL, 1.77 mmol) was added. The stirring was continued for 1 h at 0° C. and the reaction was quenched with ice-water and the mixture was extracted with CH₂Cl₂. The combined extracts were dried (Na₂SO₄) and concentrated and the residue was purified by chromatography to give the sub-title compound (0.36 g, 99%).

(d) tert-Butyl 1-(methoxymethyl)-7-azabicyclo[2.2.1]heptane-7-carboxylate

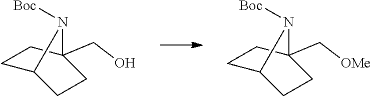 [SMILES: CC(C)(C)OC(=O)N1C2CCC1(CO)CC2 ; COCC12CCC(CC1)N2C(=O)OC(C)(C)C]

A solution of tert-butyl 1-(hydroxymethyl)-7-azabicyclo[2.2.1]heptane-7-carboxylate (0.19 g, 0.84 mmol) in THF (3 mL) was added to a stirred ice-cooled suspension of NaH (60% suspension in mineral oil, 0.10 g, 2.51 mmol, pre-washed with pentane) in THF (5 mL). The mixture was stirred at 0° C. for 30 min and MeI (0.10 mL, 1.67 mmol) was added. The ice-bath was removed and the mixture was stirred at rt for 16 h. H₂O was added and the mixture was extracted with CH₂Cl₂. The combined extracts were dried (Na₂SO₄) and carefully concentrated (the product is volatile) and the residue was purified by chromatography to give the sub-title compound (0.18 mg, 89%).

(e) tert-Butyl 1-((S)-(3-fluorophenyl)(hydroxy)methyl)-4-(methoxymethyl)-7-azabicyclo[2.2.1]heptane-7-carboxylate and tert-butyl 1-((R)-(3-fluorophenyl) -(hydroxy)methyl)-4-(methoxymethyl)-7-azabicyclo[2.2.1]heptane-7-carboxylate

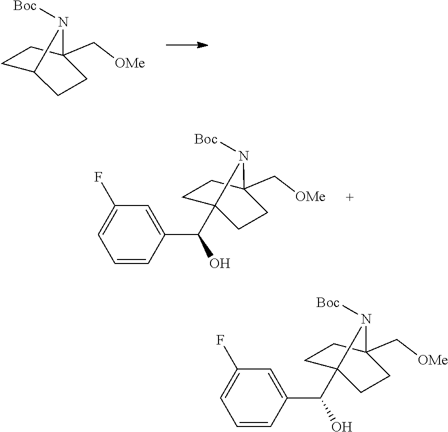 [SMILES: COCC12CCC(CC1)N2C(=O)OC(C)(C)C ; COCC12CCC([C@@H](O)c3cccc(F)c3)(CC1)N2C(=O)OC(C)(C)C ; COCC12CCC([C@H](O)c3cccc(F)c3)(CC1)N2C(=O)OC(C)(C)C]

sec-BuLi (1.23 M in cyclohexane/hexane (92/8), 0.84 mL, 1.09 mmol) was added to a solution of tert-butyl 1-(methoxymethyl)-7-azabicyclo[2.2.1]heptane-7-carboxylate (0.18 g, 0.73 mmol) and TMEDA (0.16 mL, 1.09 mmol) in Et₂O (2 mL) at −78° C. The mixture was stirred ar at −78° C. for 1 h and 3-fluorobenzaldehyde (0.15 ml, 1.45 mmol) was slowly added. The cooling bath was removed and the mixture was stirred at rt for 2 h and quenched with NH₄Cl (aq, sat) and extracted with CH₂Cl₂. The combined extracts were dried (Na₂SO₄) and concentrated. The residue was purified by chromatography to give a mixture of enantiomers, which were separated by preparative chiral HPLC (Daicel IF column, 250×30 mm, 5 μm, heptane:iPrOH=9:1, followed by heptane:iPrOH=8:2, 40 mL/min, λ 254 nm) to give the sub-title (S) enantiomer (54 mg, 20%) and the subtitle (R) enantiomer (70 mg, 26%).

(f) (S)-(3-Fluorophenyl)(4-(methoxymethyl)-7-azabicyclo[2.2.1]heptan-1-yl) -methanol hydrochloride

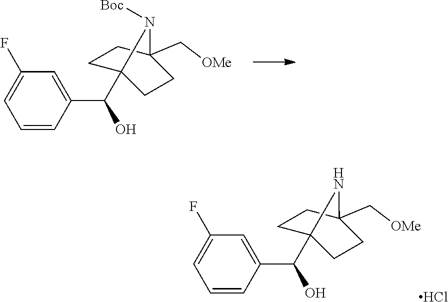 [SMILES: COCC12CCC([C@@H](O)c3cccc(F)c3)(CC1)N2C(=O)OC(C)(C)C ; COCC12CCC([C@@H](O)c3cccc(F)c3)(CC1)N2 ; Cl]

NaOH (54 mg, 1.34 mmol) in H₂O (1.7 mL) was added to a solution of tert-butyl 1-((S)-(3-fluorophenyl)(hydroxy)methyl)-4-(methoxymethyl)-7-azabicyclo[2.2.1]-heptane-7-carboxylate (49 mg, 0.13 mmol) in EtOH (1.7 mL) at rt and the mixture was heated at 120° C. for 16 h, cooled to rt and concentrated. The residue was extracted with EtOAc and the combined extracts were washed with H₂O and brine, dried (Na₂SO₄) and concentrated. The residue was dissolved in Et₂O (3 mL) and HCl (2 M in Et₂O, 87 μL, 0.18 mmol) was added dropwise at rt. The mixture was stirred at rt for 15 min and the precipitate was collected, washed with Et₂O and dried to give the title compound (34 mg, 84%).

[α]²⁵_{D}=+7.34 (c=1.09, MeOH).
¹H NMR (400 MHz, D₂O) 5 7.53-7.39 (m, 1H), 7.33-7.10 (m, 3H), 5.12 (s, 1H), 3.75 (s, 2H), 3.45 (s, 3H), 2.42-2.24 (m, 1H), 2.21-1.94 (m, 3H), 1.94-1.76 (m, 2H), 1.76-1.57 (m, 2H).

### Example 16: (R)-(3-Fluorophenyl)(4-(methoxymethyl)-7-azabicyclo[2.2.1]heptan -1-yl)methanol hydrochloride

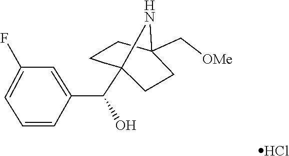 [SMILES: COCC12CCCCC([C@H](O)c3cccc(F)c3)(CC1)N2 ; Cl]

The title compound was prepared from tert-butyl 1-((S)-(3-fluorophenyl)(hydroxy)-methyl) -4-(methoxymethyl)-7-azabicyclo[2.2.1]heptane-7-carboxylate, see Example 15, Step (e) in accordance with the procedure in Example 15, Step (f).

[α]_{D}²⁵=−14.85 (c=1.01, MeOH).

The ¹H NMR spectrum was identical to the one for the (S)-enantiomer.

### Example 17: (S)-(4-((4-Chlorophenoxy)methyl)-7-azabicyclo[2.2.1]heptan -1-yl)(3-fluorophenyl)methanol

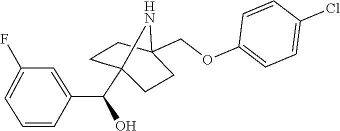 [SMILES: O[C@@H](c1cccc(F)c1)C12CCCCC(COc3ccc(Cl)cc3)(CC1)N2]

(a) tert-Butyl 1-(((tert-butyldimethylsilypoxy)methyl)-7-azabicyclo[2.2.1]heptane -7-carboxylate

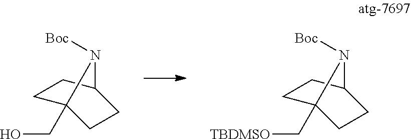 [SMILES: CC(C)(C)OC(=O)N1C2CCC1(CO)CC2 ; CC(C)(C)OC(=O)N1C2CCC1(CO[Si](C)(C)C(C)(C)C)CC2]

tert-Butyldimethylsilyl chloride (428 mg, 2.84 mmol) followed by imidazole (232 mg, 3.41 mmol) were added to a solution of tert-butyl 1-(hydroxymethyl)-7-azabicyclo-[2.2.1]heptane-7-carboxylate, see Example 1, Step (c), (430 mg, 1.89 mmol) in DMF at rt. The mixture was stirred at rt for 16 h. H₂O was added and the mixture was extracted with Et₂O. The combined extracts were washed with brine, dried (MgSO₄) and concentrated and the residue was purified by chromatography to give the subtitle compound (635 mg, 98%).

(b) 7-(tert-Butoxycarbonyl)-4-(((tert-butyldimethylsilypoxy)methyl)-7-azabicyclo -[2.2.1]heptane-1-carboxylic acid

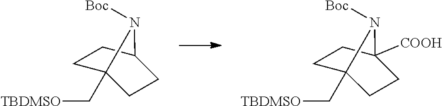 [SMILES: CC(C)(C)OC(=O)N1C2CCC1(CO[Si](C)(C)C(C)(C)C)CC2 ; CC(C)(C)OC(=O)N1C2(CO[Si](C)(C)C(C)(C)C)CCC1(C(=O)O)CC2]

sec-BuLi (1.3 M in cyclohexane, 1.35 mL, 1.76 mmol) was added dropwise to a stirred mixture of tert-butyl 1-(((tert-butyldimethylsilypoxy)methyl)-7-azabicyclo[2.2.1]-heptane -7-carboxylate (400 mg, 1.17 mmol), TMEDA (263 μL, 1.76 mmol) and Et₂O (4 mL) at −78° C. The mixture was stirred at −78° C. for 1 h and CO₂(g) was passed through the mixture for 5 min. The cooling bath was removed and the mixture was stirred at rt for 1 h. NH₄Cl (aq, sat) was added, the layers were separated and the aq phase was extracted with CH₂Cl₂. The combined organic phases were washed with brine, (Na₂SO₄) and concentrated. The residue was suspended in H₂O (5 mL) and NaOH (aq, 1 M, 1.3 mL) was added. H₂O was added until the precipitate dissolved, and the mixture was washed with Et₂O. The layers were separated and the pH of the aq phase was adjusted to 3 with KHSO₄ (aq, 2.6 M) and extracted with CH₂Cl₂. The combined extracts were washed with brine, dried (Na₂SO₄) and concentrated to give the sub-title compound (372 mg, 82%).

(c) 7-(tert-Butyl) 1-methyl 4-(((tert-butyldimethylsilyl)oxy)methyl)-7-azabicyclo -[2.2.1]heptane-1,7-dicarboxylate

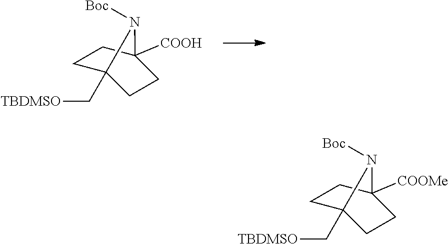 [SMILES: CC(C)(C)OC(=O)N1C2(CO[Si](C)(C)C(C)(C)C)CCC1(C(=O)O)CC2 ; COC(=O)C12CCC(CO[Si](C)(C)C(C)(C)C)(CC1)N2C(=O)OC(C)(C)C]

K₂CO₃ (grinded and then dried at 120° C., 430 mg, 3.1 mmol) was added in portions to a solution of tert-butyl 1-(((tert-butyldimethylsilypoxy)methyl)-7-azabicyclo[2.2.1]-heptane-7-carboxylate (300 mg, 0.78 mmol) in DMF (3.5 mL) at rt. The mixture was stirred at rt for 15 min and MeI (73 μL, 3.1 mmol) was added. The mixture was stirred at rt overnight. H₂O was added and the mixture was extracted with EtOAc. The combined extracts were washed with brine, dried (MgSO₄) and concentrated to give the sub-title compound (635 mg, 98%), which was used in the next step without further purification.

(d) tert-Butyl 1-(((tert-butyldimethylsilypoxy)methyl)-4-(hydroxymethyl)-7-azabicyclo[2.2.1]heptane-7-carboxylate

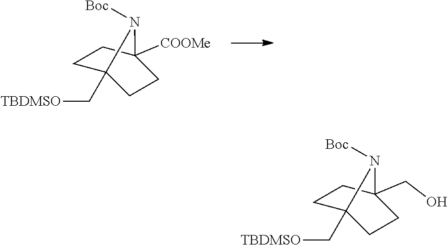 [SMILES: COC(=O)C12CCC(CO[Si](C)(C)C(C)(C)C)(CC1)N2C(=O)OC(C)(C)C ; CC(C)(C)OC(=O)N1C2(CO)CCC1(CO[Si](C)(C)C(C)(C)C)CC2]

Lithium triethylborohydride (1 M in THF, 3.8 mL, 3.8 mmol) was added dropwise to a stirred ice-cooled solution of 7-(tert-butyl) 1-methyl 4-(((tert-butyldimethylsilyl) -oxy)methyl)-7-azabicyclo[2.2.1]heptane-1,7-dicarboxylate (300 mg, 0.75 mmol) in THF (8 mL). The mixture was stirred at 0° C. for 2 h. H₂O and ice were added and the mixture was extracted with EtOAc. The combined extracts were dried (Na₂SO₄) and concentrated and the residue was purified by chromatography to give the sub-title compound (225 mg, 81%).

(e) tert-Butyl 1-(((tert-butyldimethylsilypoxy)methyl)-4-((4-chlorophenoxy)methyl) -7-azabicyclo[2.2.1]heptane-7-carboxylate

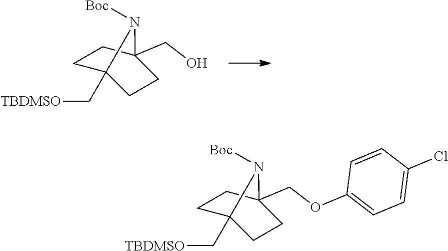 [SMILES: CC(C)(C)OC(=O)N1C2(CO)CCC1(CO[Si](C)(C)C(C)(C)C)CC2 ; CC(C)(C)OC(=O)N1C2(COc3ccc(Cl)cc3)CCC1(CO[Si](C)(C)C(C)(C)C)CC2]

4-Chlorophenol (98 mg, 0.76 mmol) and triphenyl phosphine (199 mg, 0.76 mmol) were added to an ice-cooled solution of tert-butyl 1-(((tert-butyldimethylsilyl)-oxy)methyl) -4-(hydroxymethyl)-7-azabicyclo[2.2.1]heptane-7-carboxylate (188 mg, 0.51 mmol) in THF (6 mL). After 5 min a solution of diisopropyl azodicarboxylate (156 μL, 0.76 mmol) in THF(1 mL) was added dropwise. The mixture was allowed to warm to rt and stirred at rt for 2 days. The mixture was diluted with methyl tert-butyl ether and washed with NaOH (aq, 1 M), brine, dried (Na₂SO₄) and concentrated, and the residue was purified by chromatography to give the sub-title compound (172 mg, 71%).

(f) tert-Butyl 1-((4-chlorophenoxy)methyl)-4-(hydroxymethyl)-7-azabicyclo[2.2.1]-heptane-7-carboxylate

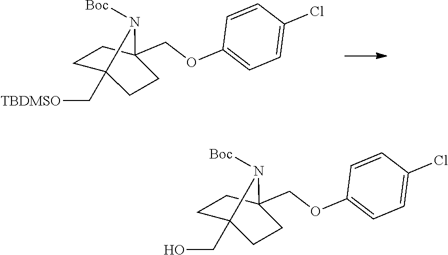 [SMILES: CC(C)(C)OC(=O)N1C2(COc3ccc(Cl)cc3)CCC1(CO[Si](C)(C)C(C)(C)C)CC2 ; CC(C)(C)OC(=O)N1C2(CO)CCC1(COc1ccc(Cl)cc1)CC2]

Tetrabutylammonium fluoride (1 M in THF, 0.94 mmol, 0.94 mL) was added dropwise to an ice-cooled stirred solution of tert-butyl 1-(((tert-butyldimethylsilypoxy)methyl) -4-(hydroxymethyl)-7-azabicyclo[2.2.1]heptane-7-carboxylate (150 mg, 0.31 mmol) in to THF (10 mL). The mixture was stirred at rt for 16 h, diluted with Et₂O, washed with H₂O and brine dried (Na₂SO and concentrated. The residue was purified by chromatography to give the subtitle compound (110 mg, 96%).

(g) tert-Butyl 1-((4-chlorophenoxy)methyl)-4-formyl-7-azabicyclo[2.2.1]heptane-7-carboxylate

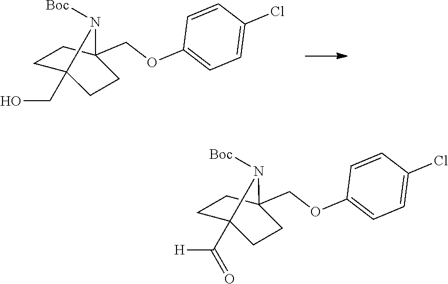 [SMILES: CC(C)(C)OC(=O)N1C2(CO)CCC1(COc1ccc(Cl)cc1)CC2 ; CC(C)(C)OC(=O)N1C2(C=O)CCC1(COc1ccc(Cl)cc1)CC2]

A solution of DMSO (48 μL, 0.68 mmol) in CH₂Cl₂ (0.5 mL) was added dropwise to a solution of oxalyl chloride (33 μL, 0.38 mmol) in CH₂Cl₂ (0.5 mL) at −78° C. for 30 min. A solution of tert-butyl 1-((4-chlorophenoxy)methyl)-4-(hydroxymethyl)-7-azabicyclo-[2.2.1]heptane -7-carboxylate (100 mg, 0.27 mmol) in CH₂Cl₂ (0.5 mL) was added dropwise and the mixture was stirred at −78° C. for 30 min. Triethylamine (0.19 mL, 1.36 mmol) was added dropwise and the mixture was stirred at −78° C. for 5 min, at 0° C. for 1 h and at rt for 30 min. H₂O was added and the layers were separated. The aq phase was extracted with CH₂Cl₂ and the combined organic phases were washed with brine, dried (MgSO₄) and concentrated. The residue was dissolved in Et₂O and filtered through Celite, which was washed with Et₂O. The filtrates were concentrated to give the sub-title compound (92 mg, 92%).

(i) tert-Butyl 1-((4-chlorophenoxy)methyl)-4-((S)-(3-fluorophenyl)(hydroxy)methyl) -7-azabicyclo[2.2.1]heptane-7-carboxylate and tert-butyl 1-((4-chlorophenoxy-methyl) -4-((R)-(3-fluorophenyl)(hydroxy)methyl)-7-azabicyclo[2.2.1]heptane-7-carboxylate

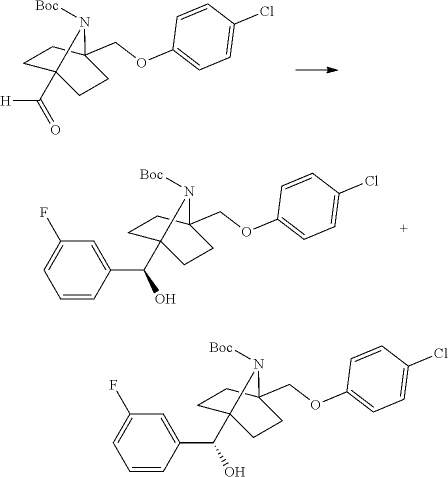 [SMILES: CC(C)(C)OC(=O)N1C2(C=O)CCC1(COc1ccc(Cl)cc1)CC2 ; CC(C)(C)OC(=O)N1C2(COc3ccc(Cl)cc3)CCC1([C@@H](O)c1cccc(F)c1)CC2 ; CC(C)(C)OC(=O)N1C2(COc3ccc(Cl)cc3)CCC1([C@H](O)c1cccc(F)c1)CC2]

3-Fluorophenylmagnesium bromide (0.9 M in THF, 535 μL, 0.48 mmol) was added dropwise to a stirred solution of tert-butyl 1-((4-chlorophenoxy)methyl)-4-formyl-7-azabicyclo[2.2.1]heptane -7-carboxylate (88 mg, 0.24 mmol) in THF (2 mL) at −78° C. and the mixture was stirred at −78° C. for 90 min. NH₄Cl (aq, sat) was added and the mixture was extracted with Et₂O. The combined extracts were washed with brine, dried (MgSO₄) and concentrated. The residue was purified by chromatography to give a mixture of enantiomers, which were separated by preparative chiral HPLC (Daicel ID column, 250×30 mm, 5 μm, heptane:iPrOH=9:1, followed by heptane:iPrOH=8:2, 40 mL/min, λ 254 nm) to give the subtitle (S) enantiomer (34 mg, 31%) and the subtitle (R) enantiomer (40 mg, 36%).

(j) (5)-(4-((4-Chlorophenoxy)methyl)-7-azabicyclo[2.2.1]heptan-1-yl)(3-fluoro-phenyl)methanol

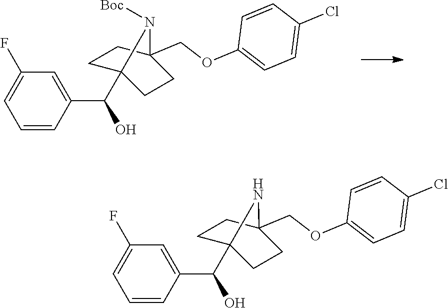 [SMILES: CC(C)(C)OC(=O)N1C2(COc3ccc(Cl)cc3)CCC1([C@@H](O)c1cccc(F)c1)CC2 ; O[C@@H](c1cccc(F)c1)C12CCC(COc3ccc(Cl)cc3)(CC1)N2]

2,6-Lutidine (151 μL, 1.3 mmol) and trimethylsilyl trifluoromethanesulfonate (59 μL, 0.33 mmol) were added to a solution of tert-butyl 1-((4-chlorophenoxy)methyl)-4-((S)-(3-fluorophenyl)(hydroxy)methyl)-7-azabicyclo[2.2.1]heptane-7-carboxylate (34 mg, 0.065 mmol) in CH₂Cl₂ (1.5 mL) at rt and the mixture was stirred at rt for 16 h. NaOH (aq, 1 M, 2 mL) was added and the layers were separated. The aq phase was extracted with CH₂Cl₂and the combined organic phases were dried (Na₂SO₄) and concentrated. The residue was dissolved in MeOH (2 mL) and NH₄F (36 mg, 0.97 mmol) was added at rt and the mixture was stirred at rt for 1 h and concentrated. The residue was dissolved in H₂O (1 mL), NaOH (aq, 1 M, 1 mL) was added and the layers were separated. The aq phase was extracted with EtOAcand the combined organic phases were washed with brine, dried (Na₂SO₄) and concentrated. The residue was dissolved in CH₂Cl₂ (2 mL) and filtered through a plug of amino-functionalized silica-gel and concentrated to give the title compound (19 mg, 81%).

[α]_{D}²⁰=+15.0 (c=0.56, CHCl₃)).
¹NMR (400 MHz, CDCl₃) δ 7.31-7.20 (m, 3H), 7.16-7.09 (m, 2H), 7.00-6.93 (m, 1H), 6.87-6.81 (m, 2H), 4.95 (s, 1H), 4.08 (s, 2H), 1.98 (ddt, J=12.2, 7.8, 3.9 Hz, 1H), 1.84 (ddt, J =15.6, 7.5, 3.7 Hz, 1H), 1.78-1.65 (m, 2H), 1.63-1.47 (m, 2H), 1.39 (ddd, J=12.9, 9.3, 4.1 Hz, 1H), 1.31-1.18 (m, 1H).

Example 18: (R)-(4-((4-Chlorophenoxy)methyl)-7-azabicyclo[2.2.1]heptan-1-yl) (3-fluorophenyl)methanol

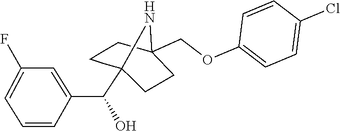 [SMILES: O[C@H](c1cccc(F)c1)C12CCCCC(COc3ccc(Cl)cc3)(CC1)N2]

The title compound was prepared from tert-butyl 1-((4-chlorophenoxymethyl)-4-((R) -(3-fluorophenyl)(hydroxy)methyl)-7-azabicyclo[2.2.1]heptane-7-carboxylate, see Example 17, Step (i) in accordance with the procedure in Example 17, Step (j)

[α]_{D}²⁰−9.8 (c=0.41, CHCl₃).

The ¹H NMR spectrum was identical to the one for the (S)-enantiomer.

### Biological Examples

L6-myoblasts were grown in Dulbecco's Modified Eagle's Medium (DMEM) containing 4.5 g/l glucose supplemented with 10% fetal bovine serum, 2 mM L-Glutamine, 50 U/ml penicillin, 50 1.1g/ml streptomycin and 10 mM HEPES. Cells were plated at 1×10⁵ cells per ml in 24-well plates. After reaching 90% confluence the cells were grown in medium containing 2% FBS for 7 days where upon cells differentiated into myotubes.

### Biological Example 1: Glucose Uptake

Differentiated L6-myotubes were serum-starved over night in medium containing 0.5% fatty-acid free BSA and stimulated with agonist, final concentration 1×10⁻⁵. After 1 h 40 min cells were washed with warm, glucose free medium or PBS and another portion of agonist was added to glucose free medium. After 20 min the cells were exposed to 50 nM ³H-2-deoxy-glucose for another 10 min before washed in ice cold glucose free medium or PBS and lysed in 0.2 M NaOH for 1 h in 60° C. Cell lysate was mixed with scintillation buffer (Emulsifier Safe, Perkin Elmer and radioactivity detected in a β-counter (Tri-Carb 2800TR, Perkin Elmer). The activity for each compound is compared to that of isoproterenol. If a compound shows activity of more than 75% of that of isoproterenol, the activity is denoted with +++, if it is between 75 and 50% it is denoted with ++; if it is between 50 and 25% it is denoted with +; if it less than 25% it is denoted with −.

### Biological Example 2: Measurement of Intracellular cAMP Levels

Differentiated cells were serum-starved over night and stimulated with agonist, final concentration 1×10⁻⁵ M, for 15 min in stimulation buffer (HBSS supplemented with 1% BSA, 5 mM HEPES and 1 mM IBMX, pH 7,4) The medium was then aspirated and to end the reaction 100 μL of 95% EtOH was added to each well of a 24-well plate and cells were kept in °20° C. over night. The EtOH was let to evaporate and 5001 μof lysis buffer (1% BSA, 5 mM HEPES and 0.3% Tween-20, pH 7,4) was added to each well before put in −80° C. for 30 min and then kept in −20° C. Intracellular cAMP levels were detected using an alpha screen cAMP kit (6760635D from Perkin Elmer). The activity for each compound is compared to that of isoproterenol. If a compound shows activity of more than 75% of that of isoproterenol, the activity is denoted with +++, if it is between 75 and 50% it is denoted with ++; if it is between 50 and 25% it is denoted with +; if it less than 25% it is denoted with −.

### Biological Example 3: Glucose Uptake in the Presence of the β₂-antagonist ICI-118,551

Differentiated L6-myotubes were serum-starved overnight in medium containing 0.5% fatty-acid free BSA and were incubated with the β₂-adrenergic receptor antagonist ICI-118,551 at a final concentration of 1×10⁻⁵ M for 30 min. The cells were stimulated with a compound of the invention, at a final concentration of 1×10⁻⁵ M. After 1 h 40 min the cells were washed twice with warm, glucose free medium or PBS and additional portions of the compound of the invention and the antagonist were added. After 20 min the cells were exposed to 50 nM ³H-2-deoxyglucose for 10 min before washed with ice cold glucose free medium or PBS three times and lysed with 0.2 M NaOH, 400 μL/well, for 1 h at 60° C. The cell lysate was mixed with 4 mL scintillation buffer (Emulsifier Safe, Perkin Elmer) and the radioactivity was detected in a β-counter (Tri-Carb 4810TR, Perkin Elmer). The activity for each compound is compared to that of isoproterenol. If a compound shows activity of more than 75% of that of isoproterenol, the activity is denoted with +++, if it is between 75 and 50% it is denoted with ++; if it is between 50 and 25% it is denoted with +; if it less than 25% it is denoted with

Using the assays described in Biological Examples 1, 2 and 3 the following results were obtained.

| Compound | Biological | Biological | Biological |
|---|---|---|---|
| example no. | example 1 | example 2 | example 3 |
| 1 | +++ | − | − |
| 2 | +++ | − | − |
| 3 | ++ | − | |
| 4 | + | − | |
| 5 | +++ | − | |
| 6 | + | − | |
| 7 | +++ | − | |
| 8 | + | − | |
| 9 | +++ | − | |
| 10 | ++ | − | |
| 11 | − | − | |
| 12 | − | − | |
| 13 | ++ | + | |
| 14 | − | − | |
| 15 | ++ | − | |
| 16 | +++ | − | − |
| 17 | ++ | − | |
| 18 | +++ | − | |

1. A compound of formula I 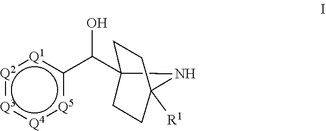
or a pharmaceutically acceptable salt thereof, wherein:
the ring comprising Q¹ to Q⁵ represents:
a phenyl optionally substituted with one or more X¹; or
a 5- or 6-membered heteroaryl optionally substituted with one or more X²;
each X¹ and X² independently represents halo, R^{a}, —CN, —N₃, —N(R^{b})R^{c}, —NO₂, —ONO₂,
—OR^{d}, —S(O)ₚR^{e} or —S(O)_{q}N(R^{f}R^{g});
each R^{a} and R^{e} represents C₁₋₆ alkyl, C₂₋₆ alkenyl or C₂₋₆ alkynyl optionally substituted by one or more groups independently selected from G¹;
each R^{b}, R^{c}, R^{d}, R^{f} and R^{g} independently represents H or C₁₋₆ alkyl, C₂₋₆ alkenyl or
C₂₋₆ alkynyl optionally substituted by one or more groups independently selected from G²;
or alternatively any of R^{b} and R^{c} and/or R^{f} and R^{g} may be linked together to form, together with the nitrogen atom to which they are attached, a 4- to 6-membered ring, which ring optionally contains one further heteroatom and which ring optionally is substituted by one or more groups independently selected from halo, C₁₋₃ alkyl optionally substituted by one or more halo, and ═O;
R¹ represents linear or branched C₁₋₁₂ alkyl, linear or branched C₂₋₁₂ alkenyl or linear or branched C₂₋₁₂ alkynyl, each optionally substituted by one G³;
each G¹and G² independently represents halo, —CN, —N(R^{a1})R^{b1}, —OR^{c1}, —S(O)ₚR^{d1},
—S(O)_{q}N(R^{e1})R^{f1} or ═O;
G³ represents —OR^{h1};
each R^{a1}, R^{b1}, R^{c1}, R^{e1} and R^{f1} independently represents H, or C₁₋₆ alkyl, C₂₋₆ alkenyl or C₂₋₆ alkynyl optionally substituted by one or more halo;
R^{d1} represents C₁₋₆ alkyl, C₂₋₆ alkenyl or C₂₋₆ alkynyl optionally substituted by one or more halo;
or alternatively any of R^{a1} and R^{b1} and/or R^{e1} and R^{f1} may be linked together to form, together with the nitrogen atom to which they are attached, a 4- to 6-membered ring, which ring optionally contains one further heteroatom and which ring optionally is substituted by one or more groups independently selected from halo, C₁₋₃ alkyl optionally substituted by one or more halo, and ═O;
Rh^{h1} represents
C₁₋₆ alkyl, or
aryl or heteroaryl optionally substituted by one or more halo;
each p independently represents 0, 1 or 2; and
each q independently represents 1 or 2.

2. A compound as defined in claim 1, wherein the compound of formula I is a compound of formula IA 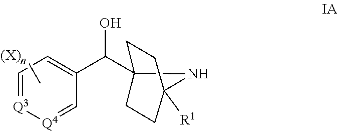
Q³ and Q⁴ each represent N or C;
when Q³ and Q⁴ both represent C, each X independently represents X¹;
when one or more of Q³ and Q⁴ represent N, each X independently represents X²; and
n represents 0 to 3.

3. A compound as defined in claim 1 or claim 2, wherein R¹ represents linear or branched C₁₋₆ alkyl optionally substituted by one G³.

4. A compound as defined in any one of the preceding claims, wherein R¹ represents a C₁₋₃ alkyl optionally substituted by one G³.

5. A compound as defined in any one of the preceding claims, wherein R¹ is not substituted.

6. A compound as defined in any one of claims 1 to 4, wherein R¹ is substituted with one G³.

7. A compound as defined in any one of claims 1 to 4 and 6, wherein R^{h1} represents C₁₋₆ alkyl, or
phenyl optionally substituted by one or more halo.

8. A compound as defined in any one of the preceding claims, wherein each X¹ and X² independently represents halo, OH, CN, C₁₋₃ alkyl or NH₂.

9. A compound as defined in any one of the preceding claims wherein each X¹ and X² independently represents halo.

10. A compound as defined in any one of the preceding claims wherein X¹ and X² independently represents F.

11. A compound as defined in any one of the preceding claims wherein each X¹ and X² independently represents Cl.

12. A compound as defined in any one of claims 2 to 11, wherein n represents 0 to 2.

13. A compound as defined in any one of claims 2 to 12, wherein n represents 1.

14. A compound as defined in any one of the preceding claims, wherein:
Q³ represents N and Q⁴ represents C; or
Q⁴ represents C and Q³ represents N.

15. A compound as defined in any one claims 1 to 13, wherein Q³ and Q⁴ represent C.

16. A compound as defined in any one of the preceding claims, wherein each X¹ or X² represents halo.

17. A compound as defined in any one of the preceding claims, wherein the compound of formula I is a compound of formula IB 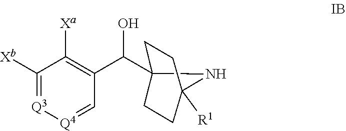
wherein:
when at least one of Q³ or Q⁴ represents N, X^{a} and X^{b} each represents H or X²;
when Q³ and Q⁴both represent C, then X^{a}and X^{b} each represents H or X¹.

18. A compound as defined in claim 17, wherein X^{a} represents H and X^{b} represents X¹ or X², as appropriate.

19. A compound as defined in claim 17, wherein X^{a} represents X¹ or X², as appropriate and X^{b} represents H.

20. A compound as defined in any one of the preceding claims for use in medicine.

21. A pharmaceutical composition comprising a compound as defined in any one of claims 1 to 19, and optionally one or more pharmaceutically acceptable adjuvant, diluent and/or carrier.

22. A compound as defined in any one of claims 1 to 19, for use in the treatment of hyperglycaemia or a disorder characterized by hyperglycaemia.

23. The use of a compound as defined in any one of claims 1 to 19, for the manufacture of a medicament for the treatment of hyperglycaemia.

24. A method of treating hyperglycaemia or a disorder characterized by hyperglycaemia comprising administering to a patient in need thereof a therapeutically effective amount of a compound as defined in any one of claims 1 to 19.

25. A pharmaceutical composition as defined in claim 21 for use in the treatment of hyperglycaemia or a disorder characterized by hyperglycaemia.

26. The compound for use, or coposition for use, method or use according to any one of claims 22 to 25, wherein the hyperglycaemia or disorder characterised by hyperglycaemia is, or is characterised by, the patient displaying severe insulin resistance.

27. The compound or compound for use, method or use according to any one of claims 22 to 26, wherein the disorder characterised by hyperglycaemia is selected from the group consisting of Type 2 diabetes, Rabson-Mendenhall syndrome, Donohue's syndrome (leprechaunism), Type A and Type B syndromes of insulin resistance, the HAIR-AN (hyperandrogenism, insulin resistance, and acanthosis nigricans) syndromes, pseudoacromegaly, and lipodystrophy.

28. A compound as defined in anyone of claims 1 to 19, for use in the treatment of a non-alcoholic fatty liver disease.

29. The use of a compound as defined in any one of claims 1 to 19, in the manufacture of a medicament for the treatment or prevention of a non-alcoholic fatty liver disease.

30. A method of treating or preenting a non-alcoholic fatty liver disease as defined in comprising administering to a patient in need thereof a therapeutically effective amount of a compound as defined in any one of claims 1 to 19.

31. A pharmaceutical composition as defined in claim 21 for use in the treatment or prevention of a non-alcoholic fatty liver disease.

32. A compound as defined in anyone of claims 1 to 19, for use in treating a disease or disorder the treatment of which is mediated by activation of the β₂ adrenergic receptor.

33. The use of a compound as defined in any one of claims 1 to 19, in the manufacture of a medicament for use in treating a disease or disorder the treatment of which is mediated by activation of the β₂ adrenergic receptor.

34. A method of treating a disease or disorder the treatment of which is mediated by activation of the β₂ adrenergic receptor comprising administering to a patient in need thereof a therapeutically effective amount of a compound as defined in any one of claims 1 to 19.

35. A pharmaceutical composition as defined in claim 21 for use in treating a disease or disorder the treatment of which is mediated by activation of the β₂ adrenergic receptor.
